(19) United States
(12) Patent Application Publication
(10) Pub. No.: US 2008/0003225 A1
(43) Pub. Date: Jan. 3, 2008

(54) Method for enhancing the antibody-dependent cellular cytotoxicity (ADCC) and uses of T cells expressing CD16 receptors

(76) Inventors: Henri Vie, Nantes (FR); Beatrice Clemenceau, Reze (FR)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/477,740

(22) Filed: Jun. 29, 2006

(51) Int. Cl. A61K 39/395; A61K 31/7072; A61K 31/704; A61K 31/66; A61K 31/56; A61K 31/525; A61K 33/24; A61K 31/282

(52) U.S. Cl. 424/144.1; 424/649; 514/49; 514/34; 514/171; 514/109; 514/251; 514/283; 514/492

(57) ABSTRACT

A method is provided for enhancing ADCC in an individual in need thereof, comprising the administration of T lymphocytes expressing a CD16-like receptor in said individual. Said method for enhancing ADCC may be used to treating cancers, autoimmune diseases or infections.

### FIELD OF THE INVENTION

The present invention relates to a method for enhancing antibody-dependent cellular cytotoxicity (ADCC), to pharmaceutical compositions comprising T cells expressing a CD16-like receptor and methods to produce or isolate T cells expressing a CD16-like receptor.

### BACKGROUND OF THE INVENTION

In the context of transplantation, donor and virus-specific T cell infusions have demonstrated the dramatic potential of T cells as immune effectors. Unfortunately, most attempts to exploit the T cell immune system against nonviral malignancies in the syngeneic setting have been disappointing. In contrast, treatments based on monoclonal antibodies (mAb) have been clinically successful. Adoptive immunotherapy with monoclonal antibodies targeting molecules such as CD20 or Her2/Neu recently have shown its capability to produce a clear clinical benefit [Glennie M J, van de Winkel J G. Drug Discov Today. 2003; 8:503-510]. Such passively acquired antibodies can trigger apoptosis of tumor cells and activate complement-mediated (CDC) or antibody-dependent cellular cytotoxicity (ADCC) in treated patients. For rituximab, an anti-CD20 humanized mAb, several clinical observations suggested that ADCC mediated by FcγRIIIa (CD16)-bearing cells is a key mechanism of action. For the anti-Her2/Neu humanized mAb trastuzumab, which is widely used to treat Her2/neu+ breast cancer, mechanisms thought to be responsible for the antitumor activity include down-modulation of the receptor, an anti-angiogenic effect, complement-dependent cytotoxicity, a direct apoptotic effect and ADCC. In fact, in a recent pilot study to elucidate the mechanism by which trastuzumab mediates its antitumor effect, R. Gennari et al observed that patients with complete or partial remission had a higher in situ leukocyte infiltration and a higher capacity to mediate in vitro ADCC [Gennari R, Menard S, Fagnoni F, et al. Clin Cancer Res. 2004; 10:5650-5655]. The findings of these clinical studies thus suggest that cancer patients eligible for mAb treatment are likely to benefit from efforts to optimize ADCC in vivo. Several effectors from both the innate and the adaptive immune system express CD16 receptors, including neutrophils, monocytes, a subset of natural killer (NK) cells, and rare T cells. Though each cell type is theoretically capable of ADCC, essentially all ADCC function in vitro was initially shown to be contained within a small fraction of cells expressing CD16 [Lanier L L, Le A M, Phillips J H, Warner N L, Babcock G F. J. Immunol. 1983; 131:1789-1796; Rumpold H, Kraft D, Obexer G, Bock G, Gebhart W. J. Immunol. 1982; 129:1458-1464; Perussia B, Starr S, Abraham S, Fanning V, Trinchieri G. Human J Immunol. 1983; 130:2133-2141; Perussia B, Trinchieri G, Jackson A, et al. J Immunol. 1984; 133:180-189; Kipps T J, Parham P, Punt J, Herzenberg L A. J Exp Med. 1985; 161:1-17].

Given i) the potential in vivo efficiency of TCRαβ T cells and the knowledge concerning their re-infusion ii) the established pertinence of several antigens such as CD20 or Her2/neu as therapeutic targets and iii) the likely influence of the ADCC pathway on the therapeutic efficiency of mAb treatments, the present invention aims to provide means for improving ADCC and therefore the efficiency of mAb treatment in vivo.

In human NK cells, FcγRIIIA (CD16) associates mainly with ITAM-containing homo-heterodimers of CD3ζ and FcεRIγ [Lanier L L, Yu G, Phillips J H. Nature. 1989; 342:803-805]. Accordingly, a FcγRIIIa/FcεRIγ fusion protein was shown to elicit intracellular responses after transfection into the Jurkat cell line. Indeed, Wirthmueller et al. [Wirthmueller U, Kurosaki T, Murakami M S, Ravetch J V. J Exp Med. 1992; 175:1381-1390.] established stable expression plasmid for FcγRIIIA+γ and used it to transfect Fc-R deficient human leukemic T cell line Jurkat. Stable expression plasmids for CD16 receptor, and CD16/ζ and CD16/γ chimeric receptor were constructed by Vivier et al. [Vivier E, Rochet N, Ackerly M, et al. Int Immunol. 1992; 4:1313-1323] and used to transfect T cell line Jurkat. Both studies found that distinct CD16 receptor isoforms reconstituted in Jurkat cells were functional for Ca²⁺ influx, IL-2 production and protein tyrosine kinase activation.

Although a Jurkat T cell line transfected with a CD16 receptor or CD16 chimeric receptor was already known in the art, the fact that the transfection of these cells with a CD16 receptor could be responsible for ADCC has not been considered nor tested. Thus, the present invention aims to provide means for enhancing ADCC and therefore the efficiency of mAb treatment in an individual in needs thereof, said means comprising the infusion of effector T cells expressing CD16 receptors in an individual.

### SUMMARY OF THE INVENTION

Therefore, it is an object of the present invention to provide a method for enhancing ADCC in said individual, said method comprises the administration of T cells expressing a CD16-like receptor in an individual in need thereof. Another object of the invention is to provide T cell clones expressing an endogenous CD16 receptor. Another object of the present invention is also to provide modified T cells expressing an exogenous CD16-like receptor.

### DESCRIPTION OF THE FIGURES

FIG. 1: Distribution of CD16 expressing cells in peripheral blood: (A) Peripheral blood mononuclear cells were stained with antibodies to αβTCR, γδTCR and CD16. Upon analysis of gated cells, three subsets of CD16 expressing cells were identified: CD16+ NK cells, CD16+ αβT-cells and CD16+ γδ-cells. Cytometric panels refers to a representative healthy donor. (B) Analysis of the absolute number of CD16+ NK cells, CD16+ αβT-cells and CD16+γδ T-cells in the peripheral blood of 26 healthy donors.

* indicates the mean.

FIG. 2. T cells coexpressing the alpha-beta T cell receptor (αβTCR) and the CD16 receptor (FcγRIIIA) can be cloned from peripheral blood lymphocytes. The αβTCR CD16+ T-cell clone retained CD16 expression and mediated ADCC during long-term culture. (A) PBMC were stained with PE-anti-αβ antibody and PC5-anti-CD16 antibody. αβ CD16+ T-cells sorting was performed on a FACSVantage™ and cloned by limiting dilution using a non specific stimulation. Cloning efficiency were 0.75 and 0.30 (according to Poisson Distribution). (B) Upper panel: Maintenance of CD16 expression in CD16+ αβ T-cell clone. T-cell clone were analysed by flow cytometry for CD16 expression over a 2.5 month period. a=Days 28 after cloning, b=Days 27 after the first non-specific stimulation, c=Days 52 after the first non-specific stimulation, d=After freezing and thawing, 38 days after the first stimulation. (B) Lower panel: Representative CD16++αβ T-cell clone was tested for ADCC activity against 51Cr-labeled autologous BLCL, in the presence of either rituximab (anti-CD20, 0.02 μg/ml or 2 μg/ml) or herceptin (anti-HER-2, 10 μg/ml) as a negative control. Results are expressed as percentage of specific lysis (effector-to-target ratio=30:1, mean of triplicate).

FIG. 3. CD16+ αβ T-cell clone produce cytokines only when the CD16 molecule is crosslinked in the presence of mAbs and target cells. (A) The CD16+/CD8+ T cell clone #14 from donor 1 and (B) the CD16+/CD4+ T-cell clone #21 from donor 2 (which doesn't recognizes the autologous BLCL through its TCR) produced TNFα after PMA+ ionomycin stimulation (a) was activated only after CD16-crosslinking in the presence of the autologous BLCL and 0.02 or 2 μg/ml of anti-CD20 (b, c and d) but remained unstimulated by the soluble mAb at concentrations up to 1000 μg/ml (e,f,g).

FIG. 4. EBV-specific polyclonal CTLs contain CD16+ αβ T cells and mediate ADCC. EBV-specific CTLs were selected against the aulogous BLCL and stained with PE-anti-αβ antibody and PC5-anti-CD16 antibody. ADCC activity of the EBV-specific CTLs was evaluated against 51Cr-labeled allogeneic BLCL in the presence of either rituximab (anti-CD20, 2 μg/ml) or herceptin (anti-HER-2, 10 μg/ml) as negative controls. Results are expressed as percentage of specific lysis (effector-to-target ratio=30:1, mean of triplicate).

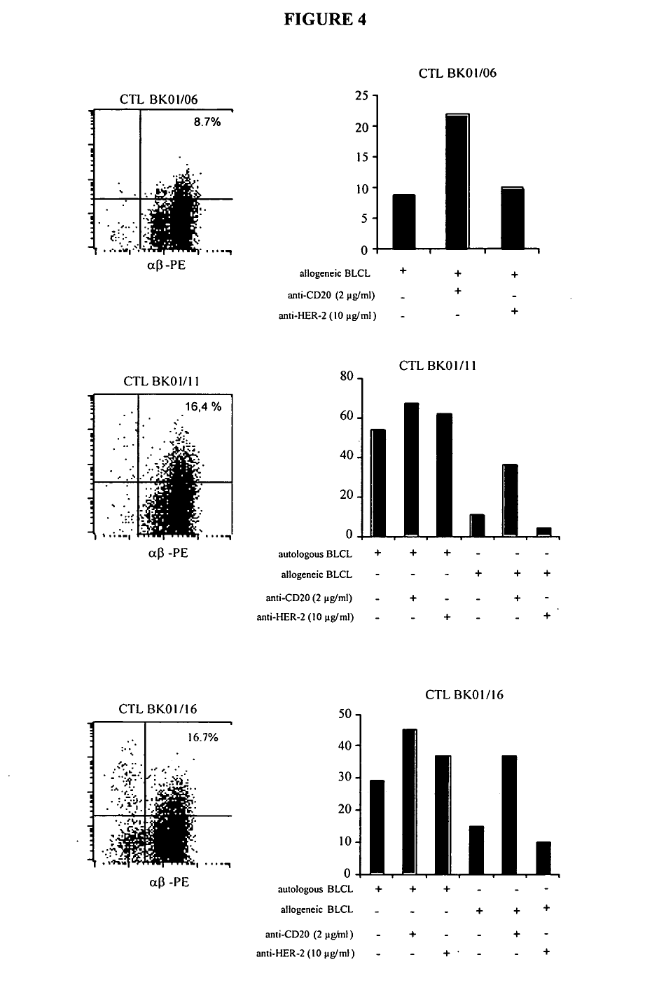

FIG. 5. (A) Schematic representation of the chimeric FcγRIIIa-FcεRIγ molecule. The CD16/γ chimeric cDNA comprised the Leader (L) and the extracellular (EC) domain of CD16 (FcγRIIIa-158V allotype), two amino-acids (aa) of the extracellular domain of the FcεRIγ as well as the intact transmembrane (TM) and intracellular (IC) domains. (B) Maintenance of chimeric receptor expression in Jurkat cells transduced with 20 μl of lentiviral virus stock. Transduced cells were analysed by flow cytometry for CD16 expression over a 3 month period. Mean fluorescence intensities are indicated in each quadrant.

FIG. 6. Generation of T cell clones expressing CD16/γ chimeric molecules. Five days after lentiviral transduction with the CD16/γ gene (MOI=10:1), the T cell clones were stained with CD16-PE (3G8) and analyzed by flow cytometry. Percentages of CD16/γ positive cells are indicated for each clone. Clone 4 and 31 are two cytolytic CD4+ HLADPB1*0401-specific T cell clones, clone 31-DO8 is a CD8+ HLA-A*0201/CMV-pp65^{N9V}-specific T-cell clone and clone 18-DO259 is a CD4+ cytolytic EBV-specific T cell clone.

FIG. 7. TCR and CD16-mediated target cell recognition by CD4+ HLA-DPB1*0401-specific cytolytic T-cell clones. Nontransduced and transduced T cell clones were tested against ⁵¹Cr-labeled HLADPB1*0401 negative or positive BLCL. ADCC activities were assessed in presence of either rituximab (anti-CD20, 2 μg/ml) or herceptin (anti-HER-2, 10 μg/ml) as negative controls. Results are expressed as percentage of specific lysis (effector-to-target ratio=30:1, mean of triplicate). For clone 31, black and white bars represent two independent experiments.

FIG. 8. Anti-CD16 mAb blocks the target cell recognition by CD16/γ transduced T-cell clones. Effector cells (the CD8+ T-cell clone #24 and the CD4+ T-cell clone #3) were first incubated in the presence or absence of anti-CD16 mAb (3G8 F(ab′)2 fragments at 20 μg/ml. After 30′ on ice effector cells were mixed (E/T ratio: 30:1) with an equal volume of ⁵¹Cr-labeled allogeneic EBV-LCL in the presence or absence of anti-CD20 mAb (rituximab, 0.2 μg/ml). Cytotoxicity was evaluated from ⁵¹Cr release after 4 h incubation, data represent mean from triplicate measurements.

FIG. 9. CD16/γ transduced T cell clone can proliferate and produce cytokines only when the CD16 molecule is crosslinked in the presence of mAbs and target cells. (A) Proliferative activity of CD16/γ transduced EBV-specific T cell clone #24 was assessed after 72-h coculture with autologous or allogeneic BLCL and IL-2 (40 IU/ml) in the presence of either rituximab (anti-CD20, 2 μg/ml) or herceptin (anti-HER-2, 10 μg/ml). Soluble anti-CD20 mAb was also tested at the higher concentrations that are indicated. (B) The CD16/γ transduced EBV-specific T cell clone #7 (which recognize through its TCR the autologous BLCL but not an allogeneic mismatch BLCL) and produced TNFα after PMA+ ionomycin stimulation (a) was activated only after CD16-crosslinking in the presence of the allogeneic BLCL and 0.02 μg/ml of anti-CD20 (b and c) but remained unstimulated by the soluble mAb at concentration 50 to 50.000 superiors (d,e,f,g).

FIG. 10. TCR and CD16 mediated target cell recognition by HLA-A*0201/CMV-pp65^{N9V}-specific C31DO8 T-cell clone. Nontransduced control and CD16/γ transduced C31DO8 T cell clones were tested (A) against an HLA-A*0201-CD20+ autologous BLCL in the presence of the increasing concentrations of N9V peptide (to test TCR-dependent cytolytic activity) and (B) in the presence of a humanized anti-CD20 mAb (rituximab) (to assess ADCC activity). Both tests were performed in the same ⁵¹Cr-release assay. Results are expressed as percent of specific lysis (effector-to-target ratio=30:1, mean of triplicate).

FIG. 11. CD8+ and CD4+ polyclonal EBV-specific CTL express CD16/γ after transduction and mediate ADCC. EBV-specific CTL were selected against the autologous BLCL and transduced with retroviral pMX-CD16/FcεRIγ according to the protocol described in the Materials and Methods section. Note in (a) that a few CD16+CD3-cells (NK cells) were present among the CTL. After transduction 14% of the CTL expressed CD16, at a level comparable to that observed in NK cells still present in the culture (b). After immunoselection and restimulation CTL were stained with CD16-PE and CD4-FITC or CD8-FITC to analyse the proportion of transduced CD4 and CD8 respectively (c). Finally a panel of CD4+ and CD8+ T cell clones was derived from the CTL to precisely assess the effect of CD16/γ transduction on the cytolytic potential of CD4 and CD8 CTLs against autologous BLCL. In (d) examples are shown of the dramatic increase in cytolytic scores observed for both the CD4+ and CD8+ clones when tested against the autologous BLCL in the presence of anti-CD20 (effector-to-target ratio=30:1). Note the example of clone CD8 n°1, which was probably not-EBV-specific, but became an effector in the presence of mAb.

### DETAILED DESCRIPTION OF THE INVENTION

I. Definitions

The term “T cell”, equivalent to “T lymphocytes”, refers to a class of lymphocytes, so called because they mature in the thymus and have the ability to recognize specific antigens through the receptors on their cell surface. T cells can be a monoclonal or polyclonal population. They can express TCRαβ or TCRγδ and CD4 or CD8 or both coreceptors, and their TCR specificity can be known or unknown.

The term “endogenous” is known in the art, and, as used herein, generally means developing or originating from within the organism or arising from causes within the organism. A T cell expressing an endogenous receptor means a T cell expressing naturally this endogenous receptor.

The term “exogenous” is known in the art, and, as used herein, generally means developing or originating from outside the organism. A T cell expressing an exogenous receptor means a T cell modified, for example by transfection or transduction, to express this exogenous receptor.

The term “transformed cell line” is known in the art, and, as used herein, generally refers to a permanently established cell culture, wherein cells are transformed and/or immortalized. For example, Jurkat cells refer to a transformed cell line derived from human T cell leukaemia.

The term “T cell clone” is known in the art, and, as used herein, generally includes T cells derived from a single T cell. T cells can be cloned using numerous methods known in the art including limiting dilution assays (LDA) and cell sorting using flow cytometry.

An “isolated” biological component (such as a nucleic acid molecule, protein, vascular tissue or haematological material, such as blood components) is known in the art, and, as used herein, generally refers to a biological component which has been substantially separated or purified away from other biological components of the cell in the organism in which the component naturally occurs. An isolated cell is one which has been substantially separated or purified away from other biological components of the organism in which the cell naturally occurs.

The term “enhance” as used herein means to improve the quality, amount, or strength of a phenomenon, especially a biological response.

The term “ADCC” or “antibody-dependent cell mediated cytotoxicity” is known in the art, and, as used herein, generally refers to a form of lymphocyte mediated cytotoxicity that functions only if antibodies are bound to the target cell. Antibody-coated target cells are killed by cells bearing Fc receptors specific for the Fc regions of the antibodies, especially NK cells.

The term “transfection” is known in the art, and, as used herein, is generally used to refer to the uptake of foreign DNA by a cell. The term “transduction” is known in the art, and, as used herein, generally denotes the delivery of a DNA molecule to a recipient cell either in vivo or in vitro, via a replication-defective viral vectors, such as retroviral gene transfer vector.

A recipient cell which has been “modified” has been generally transfected or transduced, either in vivo or in vitro, with a gene transfer vector comprising a DNA molecule of interest or with a RNA molecule of interest or with a protein of interest.

By “vector” or “gene transfer vector” is generally meant any nucleic acid construct capable of directing the expression of a gene of interest and which can transfer gene sequences to target cells. Thus, the term “vector” generally includes cloning and expression vehicles, as well as viral vectors.

By “individual”, it is meant mammal, in particular a human being.

By “effective amount”, it is meant an amount sufficient to effect a beneficial or desired clinical result (e.g. improvement in clinical condition).

As used herein, “treatment” or “treating” generally refers to a clinical intervention in an attempt to alter the natural course of the individual or cell being treated, and may be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, preventing metastasis, lowering the rate of disease progression, ameliorating or palliating the disease state, and causing remission or improved prognosis.

The term “chemotherapy” as used herein generally refers in cancer treatment to the administration of one or a combination of compounds to kill or slow the reproduction of rapidly multiplying cells. Chemotherapeutic agents include those known by those skilled in the art, including, but not limited to: 5-fluorouracil (5-FU), azathioprine, cyclophosphamide, antimetabolites (such as fludarabine), antineoplastics (such as etoposide, doxorubicin, methotrexate, and vincristine), carboplatin, cis-platinum and the taxanes, such as taxol.

The term “immuno-depleting agent” generally refers to a compound which results in a decrease in the number of cells of the immune system (such as lymphocyte) when administrated to an individual. Examples include, but are not limited to, chemotherapeutic agents.

The term “immuno-therapeutic agent” generally refers to a compound which results in the activation of an immune response when administrated to an individual. Examples include, but are not limited to, tumor antigens or monoclonal therapeutic antibodies.

II. The Present Invention

The present invention relates to a method for enhancing ADCC in an individual in need thereof, said method comprising the administration of an effective amount of T cells expressing a CD16-like receptor in said individual, wherein said CD16-like receptor is selected from the group consisting of CD16 receptors, CD16/γ chimeric receptors and CD16/ζ chimeric receptors.

T cells expressing a CD16 receptor are able to bind the constant region of antibodies via their CD16 receptor, activating by this way their mechanism of antibody-dependent cellular toxicity. Without wanting to be bound to any theory, the administration of an effective amount of T cells expressing a CD16-like receptor should increase the number of effector cells capable of activating ADCC and therefore enhance ADCC.

The term “CD16 receptor” or “CD16 receptors” refers to FcγRIIIA, isoforms of FcγRIIIA and includes fragments and variants thereof that retain CD16 biological activity.

The term “CD16 biological activity” refers to the capacity of binding IgG1 and/or IgG3 and to the capacity of mediating signals that suffice to induce immune effector functions.

The term “CD16-like receptor” refers to a cell-surface receptor which is able to bind IgG1 and/or IgG3 and is capable of mediating signals that suffice to induce immune effector functions and in particular ADCC.

The term “CD16/γ chimeric receptor” refers to a cell-surface receptor comprising the extracellular domain of CD16 (nucleotides 1 to 651, Genbank accession number No. X52645 or a sequence being substantially identical to this sequence 1-651, that is being 70% identical, preferably 80%, more preferably 90% and even more preferably 95%, 96%, 97%, 98%, 99% identical to this sequence 1-651), a transmembrane domain and the intracellular domain of the γ chain of the high affinity IgEFc receptor (FcεRIγ) (nucleotides 83 to 283, Genbank accession No BC033872, see [Vivier E, Rochet N, Ackerly M, et al. Int Immunol. 1992; 4:1313-1323], or a sequence being substantially identical to this sequence 83-283, that is being 70% identical, preferably 80%, more preferably 90% and even more preferably 95%, 96%, 97%, 98%, 99% identical to this sequence 83-283). It is understood that CD16/γ chimeric receptor includes fragments and variants that retain CD16 biological activity.

The term “CD16/ζ chimeric receptor” refers to a cell-surface receptor comprising the extracellular domain of CD16 (nucleotides 1 to 651, Genbank accession number No. X52645), a transmembrane domain and the intracellular domain of the ζ chain of the T cell antigen receptor (nucleotides 156 to 566, Genbank accession No J04132, see [Vivier E, Rochet N, Ackerly M, et al. Int Immunol. 1992; 4:1313-1323], or a sequence being substantially identical to this sequence 83-283, that is being 70% identical, preferably 80%, more preferably 90% and even more preferably 95%, 96%, 97%, 98%, 99% identical to this sequence 156-566). It is understood that CD16/ζ, chimeric receptor includes fragments and variants that retain CD16 biological activity.

It is understood that the term “extracellular domain of CD16” includes fragments and variants that retain the capacity of binding IgG1 and/or IgG3.

In one embodiment of the invention, T cells expressing a CD16-like receptor are natural T cells expressing an endogenous CD16 receptor.

In another embodiment of the invention, said T cells expressing a CD16-like receptor are modified T cells expressing an exogenous CD16-like receptor.

T cells expressing a CD16 receptor, although being present in all individuals, have been described to represent rare and very specific subsets (especially T cells in a terminal differentiation status), rendering their manipulation difficult to envisage [Uciechowski P, Werfel T, Leo R, Gessner J E, Schubert J, Schmidt R E. Immunobiology. 1992; 185:28-40; Oshimi K, Oshimi Y, Yamada O, Wada M, Hara T, Mizoguchi H. lymphocytes. J. Immunol. 1990; 144:3312-3317; Groh V, Porcelli S, Fabbi M, et al. J Exp Med. 1989; 169:1277-1294; Lafont V, Liautard J, Liautard J P, Favero J. J Immunol. 2001; 166:7190-7199; Angelini D F, Borsellino G, Poupot M, et al. Blood. 2004; 104:1801-1807.] Surprisingly, the applicant shows in the present invention that a significant population of T cells expressing an endogenous CD16 receptor is present in all individuals, can be cloned from peripheral blood lymphocytes and are capable of ADCC. In addition, the applicant shows that T cells can be modified to express an exogenous CD16-like receptor, to render these cells capable of ADCC.

In a preferred embodiment of the invention, said effective amount of T cells expressing a CD16-like receptor is administrated in an individual in need thereof via a parenteral route. A parenteral administration mode includes subcutaneous, intramuscular, intravenous, intraperitoneal, intranasal and intradermal administration. Administration can be systemic or local.

In a more preferred embodiment of the invention, said T cells expressing a CD16-like receptor are intravenously administrated in an individual in need thereof.

In another embodiment of the invention, said T cells expressing a CD16-like receptor are administrated at a dose of about 1 to 5×10⁶ cells per kilogram to about 10⁹ cells per kilogram.

Preferably, said T cells expressing a CD16-like receptor are administrated at a dose of about 10⁷ cells per kilogram to 10⁹ cells per kilogram, more preferably to about 10⁸ cells per kilogram to 10⁹ cells per kilogram.

According to the invention, said method for enhancing ADCC permits the treatment of cancers, auto-immune diseases, tissue graft or organ rejections, including graft versus host disease, and infectious diseases. Indeed ADCC plays a major role in such diseases or conditions for the elimination of infected cells, tumor cells . . . .

Certain embodiments of this invention relate to combination therapies. According to the invention, said method for enhancing ADCC further comprises the administration of at least one immuno-therapeutic agent such as tumor antigens for antitumoral vaccination or monoclonal therapeutic antibodies for monoclonal antibody therapy. The administration of T cells expressing a CD16-like receptor should indeed enhance the effect of said immuno-therapeutic agents via the enhancement of ADCC.

In one embodiment of the invention, said immuno-therapeutic agent comprises tumor antigens. Tumor antigens include but are not limited to peptides derived from the MAGE, BAGE, GAGE and LAGE1/NY-ESO-1 gene families. These tumor antigens can be administrated alone or can be presented by an antigen presenting cells such as dendritic cells or can be contain in a delivery system such as exosomes, apoptotic bodies, or tumor cells.

In another embodiment of the invention, said immuno-therapeutic agent comprises monoclonal therapeutic antibodies. Examples of monoclonal antibodies include, but are not limited to, Infliximab (anti-TNFα), Basiliximab, Daclizumab (anti-CD25), Trastuzumab (anti-Her2/neu), Rituximab, Ibritumomab tiutexan (anti-CD20), Tositumomab (anti-CD122), Gemtuzumab ozogamicin (anti-CD33), Alemtuzumab (anti-CD52). Such agents can be administrated before, during or after administration of the T cells expressing a CD16-like receptor.

According to the invention, said method for enhancing ADCC further comprises the administration of at least one immuno-depleting agent.

As shown for example in Dudley et al. Science. 2002 October 25; 298(5594):850-4 and in Nat Med. 2005 November; 11(11):1230-7, lymphodepletion can have a marked effect on the efficacy of T cell transfer therapy. Preferably, such chemotherapeutic agents are administrated before the administration of the T cells expressing a CD16-like receptor.

In one embodiment of the invention, said immuno-depleting agents comprise at least one chemotherapeutic agent. Examples of chemotherapeutic agents include, but are not limited to, 5-fluorouracil, aziathioprine, cyclophosphamide, anti-metabolites (such as fludarabine), anti-neoplastics (such as etoposide, doxorubicin, methotrexate, vincristine), prednisone, carboplatin, cis-platinum and the taxanes such as taxol.

In another embodiment of the invention, the administration of said T cells expressing a CD16-like receptor is made about 10 days to about one month after the administration of at least one immuno-therapeutic agent. Preferably, said administration is made about 10 days to about 3 weeks after the administration of at least one immuno-therapeutic agent.

However, it is understood that the regimen of administration of said T cells expressing a CD16-like receptor is within the judgment of the managing physician, and depends on the clinical condition of the individual, the objectives of treatment, and concurrent therapies also being administrated.

In another embodiment of the invention, immuno-depleting agent such as chemotherapeutic agents defined hereabove can be administrated 2 days, preferably 1 day, before the administration of T cells expressing a CD16-like receptor.

In a preferred embodiment, the method for enhancing ADCC according to the invention permits the treatment of cancer, optionally in combination with antitumoral vaccination. Said method comprises the administration in an individual in needs thereof of the T cells expressing CD16-like receptor in combination with at least one tumor antigen. Tumor antigens such as peptides derived from the MAGE, BAGE, GAGE and LAGE1/NY-ESO-1 gene families are used for treating many melanomas, transitional bladder cancers, head and neck squamous cells carcinomas, non small cell lung cancers, oesophageal cancers, multiple myelomas.

In a preferred embodiment, the method for enhancing ADCC according to the invention permits the treatment of cancer, especially solid tumors, optionally in combination with monoclonal antibody therapy. Said method comprises the administration in an individual in need thereof of the T cells expressing a CD16-like receptor in combination with at least one monoclonal antibody used for treating solid tumors.

Solid tumors, such as sarcomas and carcinomas, comprise fibrosarcoma, myxosarcoma, liposarcoma, chondrosarcoma, osteogenic sarcoma, colon carcinoma, lymphoid malignancy, pancreatic cancer, breast cancer, lung cancer, ovarian cancer, prostate cancer, renal cell carcinoma, melanoma, CNS tumors . . . .

Examples of monoclonal antibody used for treating solid tumors include but are not limited to Trastuzumab used for treating breast cancer or Rituximab, Ibritumomab tiutexan or Tositumomab for treating lymphoma.

In a preferred embodiment, the method for enhancing ADCC according to the invention permits the treatment of cancer, especially haematological tumors, optionally in combination with monoclonal antibody therapy. Said method comprises the administration in an individual in needs thereof of the T cells expressing CD16-like receptor in combination with at least one monoclonal antibody used for treating hematologic or lymphoid malignancies.

Hematological tumors comprise acute lymphocytic leukaemia, acute myelogenous leukaemia, chronic lymphocytic leukaemia, chronic myelogenous leukaemia, indolent non Hodgkin's lymphoma, high-grade Hodgkin's lymphoma, Hodgkin's lymphoma, multiple myeloma or myelodysplastic syndrome. Examples of monoclonal antibody used for treating hematologic or lymphoid malignancies include, but are not limited to, Gemtuzumab ozogamicin used for treating acute myelogenous leukaemia, or Alemtuzumab used for treating chronic lymphocytic leukaemia.

In a preferred embodiment, the method for enhancing ADCC according to the invention permits the treatment of autoimmune diseases, optionally in combination with monoclonal antibody therapy. Said method comprises the administration in an individual in need thereof of the T cells expressing a CD16-like receptor in combination with at least one monoclonal antibody used for treating autoimmune diseases. Autoimmune diseases comprise type I diabetes, multiple sclerosis, systemic lupus erythemateous, thyroiditis, rheumatoid arthritis. Examples of monoclonal antibody used for treating autoimmune diseases include but are not limited to Infliximab used for treating polyarthrite rhumatoïde or Cröhn disease.

In a preferred embodiment, the method for enhancing ADCC according to the invention permits the treatment of tissue graft or organ rejection, including graft versus host disease (GVHD), optionally in combination with monoclonal antibody therapy. Said method comprises the administration in an individual in need thereof of the T cells expressing a CD16-like receptor in combination with at least one monoclonal antibody used for treating tissue graft or organ rejection.

Grafts, referring to biological material derived from a donor for transplantation into a recipient, include such diverse material as, for example, isolated cells such as islet cells and neural-derived cells, tissue such as the amniotic membrane of a newborn, bone marrow, hematopoietic precursor cells, and organs such as skin, heart, liver, spleen, pancreas, thyroid lobe, lung, kidney, tubular organs . . . . Examples of monoclonal antibody used for treating tissue graft or organ rejection include but are not limited to Basiliximab or Daclizumab used for treating kidney rejection.

In another embodiment, the method for enhancing ADCC according to the invention permits also the treatment of infectious diseases, especially bacterial and viral infections.

It is another object of the present invention to provide a T cell clone expressing an endogenous CD16 receptor.

In a preferred embodiment, said T cell clone expressing an endogenous CD16 receptor has been isolated.

In a preferred embodiment of the invention, a T cell clone expressing an endogenous CD16 receptor expresses an antigen specific receptor (TCR) of known specificity.

Knowing the specificity of the TCR will permit to anticipate that the T cells will be unable to recognize non infected or non transformed host tissues.

Indeed, from an immunological point of view, the use of specific T cells whose TCR specificity is known should be safer than the use of a bulk population and will avoid the risk of a graft versus host reaction when allogeneic T cells are used. The specificity of the antigen specific receptor of the T cells can be defined by any methods known in the art, for example by flow cytometry, cytotoxicity assay or proliferation assay.

In one embodiment of the invention, the specificity of said T cell clone expressing an endogenous CD16 receptor is directed against a virus selected from the group consisting in Epstein Barr viruses (EBV), cytomegaloviruses (CMV), human papilloma viruses (HPV), and herpes simplex virus (HSV1, HSV2). Preferably, the specificity of said T cell clone expressing endogenous CD16 receptor is directed against EBV.

In another embodiment of the invention, the specificity of said T cell clone expressing an endogenous CD16 receptor is directed against the human leukocyte antigen system (HLA). HLA is the general name of a group of genes in the human major histocompatibility complex (MHC) region on human chromosome 6 (mouse chromosome 17) that encodes the cell-surface antigen presenting proteins. HLA molecules comprise HLA-A, HLA-B, HLA-C, HLA-DPA1, HLA-DPB1, HLA-DQA1, HLA-DQB1, HLA-DRA, and HLA-DRB1.

In a preferred embodiment of the invention, a T cell clone expressing an endogenous CD16 receptor expresses an antigen specific receptor (TCR) of known specificity, and a specific HLA combination, that is widespread in the recipient individuals. For example, the T cell clone expressing an endogenous CD16 receptor is derived from an individual being heterozygous, preferably homozygous, for the haplotype HLA A1B8DR3-DQ2 and can preferably be administrated in a Caucasian individual, for which this haplotype is widespread. Preferably, the T clone expressing an endogenous CD16 receptor is derived from an individual being homozygous for the haplotype HLA A1B8DR3-DQ2.

Indeed, from an immunological point of view, the use of an allogeneic T cell clone further expressing a specific HLA combination, being widespread in the recipient individuals should allow an increased lifetime of this clone as the T cell clone expressing said HLA combination would be less recognized as non-self by the immune system of said individual.

It is an object of the invention to provide a method for isolating said T cell clone expressing an endogenous CD16 receptor, wherein said method comprises:
isolating T cells expressing an endogenous CD16 receptor from PBL,
purifying said T cells expressing an endogenous CD16 receptor,
cloning T cells expressing an endogenous CD16 receptor,
optionally further expanding the at least one T cell clone thus obtained.

In a preferred embodiment of the invention, T cells expressing an endogenous CD16 receptor are isolated from PBL by using monoclonal antibodies and flow cytometry. T cells can be autologous or allogenic.

The isolated T cells expressing an endogenous CD16 receptor can further be substantially purified by any well known method in the art. A substantially purified population of cells refers to a population of cells that are at least 80%, 90%, 95%, 96%, 97%, 98% or 99% pure. Preferably, isolated T cells expressing an endogenous CD16 receptor are sorted by flow cytometry using anti-αβp antibody and anti-CD16 antibody. However, for clinical use of these T cell expressing an endogenous CD16 receptor, these isolated cells are purified by using immunomagnetic methods.

Purified T cells expressing an endogenous CD16 receptor are further cloned by any method well known in the art, for example by a non-specific amplification procedure described in Gaschet et al. [Gaschet et al. Blood 1996, 87:2345-2353]. Finally, T cell clones expressing an endogenous CD16 receptor are further expanded by cell culture. The expansion of the T cell clones can be realized by in vitro non specific stimulation such as those provided by exposure to CD3 and CD28 mAb or lectins such as PHA, or by specific stimulation such as those provided by coculture of T cells with allogeneic or virally infected cells or with a soluble antigen. The soluble antigen may be a peptide corresponding to a viral epitope that stimulates αβ T cells or a non-peptidic molecule capable of stimulating γδ T cells.

The specificity of the TCR of the T cell clones expressing an endogenous CD16 receptor thus obtained can be further assessed by any well-known method in the art, for example by cytotoxicity assay, cytotoxicity assay or proliferation assay.

It is also an object of the invention to provide a method for producing a T cell clone expressing an endogenous CD16 receptor, a TCR of known specificity, and optionally expressing a specific HLA combination that is widespread in the recipient individuals, comprising:
isolating and expanding at least one (known-antigen)-specific T cell optionally expressing a specific HLA combination that is widespread in the recipient individuals,
cloning said (known-antigen)-specific T cell,
isolating at least one (known-antigen)-specific T cell clone expressing an endogenous CD16 receptor,
and optionally expanding said (known-antigen)-specific T cell clone expressing an endogenous CD16 receptor.

In one embodiment, the isolation and expansion of at least one (known-antigen)-specific T cell is realized according to standard methods by stimulating PBL with said known-antigen or with a cell line expressing said known antigen. For example, the isolation and expansion of an EBV specific cytotoxic T cell is realized by stimulating PBL with an EBV B lymphoblastoid cell line (BLCL) according to standard methods. Another example of CMV specific cytotoxic T cells is described in Gallot et al. [Gallot et al., JI 2001, 167, 4196:4206].

In a preferred embodiment, the known-antigen is selected from the group consisting of EBV, CMV, HPV, HSV1 and HSV2, or is directed against HLA molecules. Said (known-antigen)-specific T cell optionally expresses a specific HLA combination, that is widespread in the recipient individuals and can be obtained by using PBL derived from an individual being heterozygous, preferably homozygous, for this specific HLA combination.

The (known-antigen)-specific T cell are further cloned by any method well known in the art, for example by a non-specific amplification procedure described in Gaschet et al. [Gaschet et al. Blood 1996, 87:2345-2353].

Among (known-antigen)-specific T cell clones thus obtained, is isolated a (known-antigen)-specific T cell clone that expresses an endogenous CD16 receptor. Such isolation can be realized by immunostaining using flow cytometry. Finally, T cell clones expressing an endogenous CD16 receptor can optionally be expanded by cell culture. The expansion of the T cell clones can be realized by in vitro non specific stimulation such as those provided by exposure to CD3 and CD28 mAb or lectins such as PHA, or by specific stimulation such as those provided by coculture of T cells with allogeneic or virally infected cells or with a soluble antigen. The soluble antigen may be a peptide corresponding to a viral epitope that stimulates αβ T cells or a non-peptidic molecule capable of stimulating γδ T cell.

Another object of the invention is to provide a pharmaceutical composition comprising at least one T cell clone expressing an endogenous CD16 receptor as described above.

In a preferred embodiment, said pharmaceutical composition includes an effective amount of T cell clone expressing an endogenous CD16 receptor, alone or with a pharmaceutically acceptable carrier.

The pharmaceutically acceptable carriers useful herein are conventional. Remington's Pharmaceutical Sciences 16^{th} edition, Osol, A. Ed. (1980) describes composition and formulations suitable for pharmaceutical delivery of the T cell clone expressing endogenous CD16 receptor herein disclosed. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as vehicle. The carrier and composition can be sterile, and the formulation suits the mode of administration. In addition to biological neutral carriers, pharmaceutical compositions to be administrated can contain minor amounts of non toxic auxiliary substances, such as wetting or emulsifying agents, preservatives, and pH buffering agents and the like, for example sodium acetate or sorbitan monolaurate. The composition can be a liquid solution, suspension, emulsion.

The amount of T cell clone expressing an endogenous CD16 receptor effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each individual's circumstances. Effective doses can be extrapolated from dose-response curves derived from in vitro or animal model test systems.

In a preferred embodiment, said pharmaceutical composition includes an effective amount of T cell clone expressing an endogenous CD16 receptor with human albumin.

In a preferred embodiment, said pharmaceutical composition is administrated in an individual in need thereof by intravenous injections.

In a preferred embodiment, said pharmaceutical composition is used for treating diseases or conditions requiring an ADCC enhancement selected from the group consisting of cancers, autoimmune diseases, tissue graft or organ rejections, bacterial or viral infections. Preferably said pharmaceutical composition is used for treating cancers.

It is also an object of the present invention to provide modified T cell expressing an exogenous CD16-like receptor, said CD16-like receptor being selected from the group consisting in CD16 receptors, CD16/γ chimeric receptors and CD16/ζ chimeric receptors.

In a preferred embodiment of the invention, the exogenous CD16-like receptor is a CD16/γ chimeric receptor.

In a preferred embodiment the extracellular domain of the CD16-like receptor comprises the extracellular domain of the allotype V¹⁵⁸ of CD16 receptor.

The gene coding FcγRIIIA displays a functional allelic dimorphism generating allotypes with either a phenylalanine (F) or a valine (V) residue at amino acid position 158. In vitro, NK cells from donors homozygous for FcγRIIIa-158V (VV) bound more human IgG1 and IgG3 than did NK cells from donors homozygous for FcγRIIIa-158F (FF) [Koene H R, Kleijer M, Algra J, Roos D, von dem Borne A E, de Haas M. Blood. 1997; 90:1109-1114]. In vivo, Cartron et al, have recently shown that the genotype homozygous for FcγRIIIa-158V (VV) is associated with a higher clinical response to rituximab in the treatment of follicular non Hodgkin's lymphomas (NHL) [Cartron G, Dacheux L, Salles G, et al. Blood. 2002; 99:754-758]. Therefore the allotype V¹⁵⁸ of CD16 should be more efficient to induce immune effector functions and in particular ADCC.

In a preferred embodiment, said modified T cell expressing an exogenous CD16-like receptor is a modified T cell clone expressing an exogenous CD16-like receptor.

In a preferred embodiment of the invention, said modified T cell clone expressing an exogenous CD16-like receptor expresses an antigen specific receptor (TCR) of known specificity.

In one embodiment of the invention, the specificity of said modified T cell clone expressing an exogenous CD16-like receptor is directed against a virus selected from the group consisting of Epstein Barr viruses (EBV), cytomegaloviruses (CMV), human papilloma viruses (HPV), and herpes simplex virus (HSV1, HSV2).

Preferably, the specificity of said modified T cell clone expressing an exogenous CD16-like receptor is directed against EBV. Preferably, the specificity of said modified T cell clone expressing an exogenous CD16-like receptor is directed against CMV.

In another embodiment of the invention, the specificity of said T cell clone expressing an exogenous CD16-like receptor is directed against the human leukocyte antigen system (HLA). HLA is the general name of a group of genes in the human major histocompatibility complex (MHC) region on human chromosome 6 (mouse chromosome 17) that encodes the cell-surface antigen presenting proteins. HLA molecules comprise HLA-A, HLA-B, HLA-C, HLA-DPA1, HLA-DPB1, HLA-DQA1, HLA-DQB1, HLA-DRA, and HLA-DRB1.

In a preferred embodiment of the invention, a T cell clone expressing an exogenous CD16-like receptor expresses an antigen specific receptor (TCR) of known specificity, and a specific HLA combination, that is widespread in the recipient individuals. For example, the T cell clone expressing an exogenous CD16-like receptor is derived from an individual heterozygous for the haplotype HLA A1B8DR3-DQ2 and can preferably be administrated in a Caucasian individual, for which this haplotype is widespread. Preferably, the T cell clone expressing an exogenous CD16-like receptor is derived from an individual homozygous for the haplotype HLA A1B8DR3-DQ2

Indeed, from an immunological point of view, the use of an allogeneic T cell clone further expressing a specific HLA combination, being widespread in the recipient individuals should allow an increased lifetime of this clone as the T cell clone expressing said HLA combination would be less recognized as non-self by the immune system of said individual.

Another object of the present invention is to provide a method for producing said modified T cells expressing an exogenous CD16-like receptor, said method comprises:
isolating T cells,
transfecting or transducing said T cells to allow expression of an exogenous CD16-like receptor,
purifying the modified T cells expressing an exogenous CD16-like receptor thus obtained,
optionally cloning the modified T cells expressing exogenous CD16-like receptor,
and optionally further expanding the T cell clones thus obtained.

According to the invention, T cells are isolated from PBL by using monoclonal antibodies and flow cytometry. T cells can be autologous or allogenic. The modified T cells expressing an exogenous CD16-like receptor can then be obtained by standard methods well known in the art. Gene delivery standard methods may be for example lipid delivery using cationic lipids, viral delivery, delivery by electroporation or delivery by others chemical methods such as calcium phosphate precipitation, DEAE-dextran, polybrene.

The T cells expressing an exogenous CD16-like receptor can further be substantially purified by any well known method in the art. A substantially purified population of cells refers to a population of cells that are at least 80%, 90%, 95%, 96%, 97%, 98% or 99% pure. Preferably, T cells expressing an exogenous CD16-like receptor are sorted by flow cytometry using anti-αβ antibody and anti-CD16 antibody. However, for clinical use, these T cell expressing an exogenous CD16-like receptor are purified by using immunomagnetic methods.

Purified T cells expressing an exogenous CD16-like receptor are further cloned by any method well known in the art, for example by a non-specific amplification procedure described in Gaschet et al. 1996. Finally, CD16 receptor expressing T cell clones are further expanded by cell culture. The expansion of the T cell clones can be realized by in vitro non specific stimulation such as those provided by exposure to CD3 and CD28 mAb or lectins such as PHA, or by specific stimulation such as those provided by coculture of T cells with allogeneic or virally infected cells or with a soluble antigen. The soluble antigen may be a peptide corresponding to a viral epitope that stimulates αβ T cells or a non-peptidic molecule capable of stimulating γδ T cell.

The specificity of the TCR of the T cell clones expressing an exogenous CD16-like receptor thus obtained can be further assessed by any well-known method in the art, for example by cytotoxicity assay.

It is also an object of the invention to provide a method for producing modified T cells expressing an exogenous CD16-like receptor, a TCR of known specificity, and optionally expressing a specific HLA combination that is widespread in the recipient individuals, comprising:
isolating and expanding at least one (known-antigen)-specific T cell optionally expressing a specific HLA combination that is widespread in the recipient individuals,
transfecting or transducing said (known-antigen)-specific T cell to allow expression of an exogenous CD16-like receptor,
isolating said (known-antigen)-specific T cell expressing an exogenous CD16-like receptor,
optionally cloning said (known-antigen)-specific T cell expressing an exogenous CD16-like receptor,
optionally purifying said (known-antigen)-specific T cell clone expressing an exogenous CD16-like receptor,
and optionally expanding said (known-antigen)-specific T cell clone expressing an exogenous CD16-like receptor.

In one embodiment, the isolation and expansion of at least one (known-antigen)-specific T cell is realized according to standard method by stimulating PBL with a cell line expressing said known antigen or with said known-antigen. For example, the generation and expansion of EBV specific cytotoxic T cells is realized by stimulating PBL with an EBV B lymphoblastoid cell line (BLCL) according to standard methods. Another example of CMV specific cytotoxic T cells is described in Gallot et al. [Gallot et al., JI 2001, 167, 4196:4206].

In a preferred embodiment, the known-antigen is selected from the group consisting of EBV, CMV, HPV, HSV1 and HSV2, or is directed against HLA molecules. Said (known-antigen)-specific T cell optionally expresses a specific HLA combination, that is widespread in the recipient individuals and can be obtained by using PBL derived from an individual being heterozygous, preferably homozygous, for this specific HLA combination.

The transfection or transduction of (known-antigen)-specific T cell is realized as described previously. (Known-antigen)-specific T cells expressing an exogenous CD16-like receptor are further optionally cloned by any method well known in the art, for example by a non-specific amplification procedure described in Gaschet et al. 1996.

Among (known-antigen)-specific T cell clones thus obtained, is isolated a (known-antigen)-specific T cell clone that expresses an exogenous CD16-like receptor. Such isolation can be realized by immunostaining using flow cytometry. Then, a known-antigen specific T cell clone expressing an exogenous CD16-like receptor can optionally be substantially purified by any well-known methods in the art. For clinical use, the purification is preferably realized by using immunomagnetic methods. Finally, T cell clones expressing an exogenous CD16-like receptor can optionally be expanded by cell culture. This expansion can be realized by in vitro non specific stimulation such as those provided by exposure to CD3 and CD28 mAb or lectins such as PHA, or by specific stimulation such as those provided by coculture of T cells with allogeneic or virally infected cells or with a soluble antigen. The soluble antigen may be a peptide corresponding to a viral epitope that stimulates αβ T cells or a non-peptidic molecule capable of stimulating γδ T cell.

In one embodiment of the invention, isolated T cells are transfected or transduced by a vector comprising a gene encoding a CD16-like receptor, said CD16-like receptor being selected from the group consisting of CD16 receptors, CD16/γ chimeric receptors and CD16/ζ chimeric receptors.

In a preferred embodiment of the invention, the vector used to transduce T cells is a viral vector, such as retrovirus, adenovirus, adenovirus associated virus, comprising a gene encoding CD16-like receptor, said CD16-like receptor being selected from the group consisting of CD16 receptors, CD16/γ chimeric receptors and CD16/ζ chimeric receptors.

Preferably, the vector used to transduce T cells is a lentivirus comprising a gene encoding a CD16-like receptor, said CD16-like receptor being selected from the group consisting of CD16 receptors, CD16/γ chimeric receptors and CD16/ζ chimeric receptors. In the art several lentiviral vectors that allow specific targeting of transgene expression to T cell have been generated (see for example Hum Gene Ther. 2006 March; 17(3):303-13, J Gene Med. 2004 September; 6(9):963-73, Hum Gene Ther. 2003 July 20; 14(11):1089-105 and Blood. 2003 May 1; 101(9):3416-23). The constructs encoding CD16 receptor, CD16/γ chimeric receptor and CD16/ζ chimeric receptor can be found in Viver et al. [Vivier E, Rochet N, Ackerly M, et al. Int Immunol. 1992; 4:1313-1323], Wirthmueller et al. [Wirthmueller U, Kurosaki T, Murakami M S, Ravetch J V. J Exp Med. 1992; 175:1381-1390.] and in the following examples.

It is also an object of the present invention to provide a viral vector comprising a gene encoding a CD16-like receptor, said CD16-like receptor being selected from the group consisting of CD16 receptors, CD16/γ chimeric receptors and CD16/ζ chimeric receptors, for producing modified T cells expressing an exogenous CD16-like receptor. Preferably, said viral vector is a lentivirus.

Another object of the invention is to provide a composition comprising at least one modified T cell expressing an exogenous CD16-like receptor, said CD16-like receptor being selected from the group consisting of CD16 receptors, CD16/γ chimeric receptors and CD16/ζ chimeric receptors.

In one embodiment, said composition comprises at least one modified T cell clone expressing an exogenous CD16-like receptor as described previously.

In another embodiment, said composition comprises at least one modified T cell clone expressing an exogenous CD16-like receptor, a TCR of known specificity and optionally a specific HLA combination that is widespread in the recipient individuals. Preferably, said composition comprises at least one modified T cell clone expressing an exogenous CD16-like receptor, which TCR specificity is directed against virus selected in the group consisting of EBV, CMV, HPV, HSV1 and HSV2, or is directed against HLA antigens.

Another object of the invention is to provide a pharmaceutical composition comprising at least one modified T cell expressing an exogenous CD16-like receptor, said CD16-like receptor being selected from the group consisting of CD16 receptors, CD16/γ chimeric receptors and CD16/ζ chimeric receptors.

In one embodiment, said pharmaceutical composition comprises at least one modified T cell clone expressing an exogenous CD16-like receptor as described previously.

In another embodiment, said pharmaceutical composition comprises at least one modified T cell clone expressing an exogenous CD16-like receptor, a TCR of known specificity and optionally a specific HLA combination that is widespread in the recipient individuals. Preferably, said composition comprises at least one modified T cell clone expressing an exogenous CD16-like receptor, which TCR specificity is directed against virus selected in the group consisting of EBV, CMV, HPV, HSV1 and HSV2, or is directed against HLA antigens.

In a preferred embodiment, said pharmaceutical composition includes an effective amount of modified T cells expressing an exogenous CD16-like receptor, alone or with a pharmaceutically acceptable carrier.

In a preferred embodiment, said pharmaceutical composition includes an effective amount of T cell clone expressing an exogenous CD16-like receptor with human albumin.

In a preferred embodiment, said pharmaceutical composition is administrated in an individual in need thereof by intravenous injections.

In a preferred embodiment, said pharmaceutical composition is used for treating diseases or conditions requiring an ADCC enhancement selected from the group consisting of cancers, autoimmune diseases, tissue graft or organ rejection, including GVHD, bacterial or viral infections. Preferably said pharmaceutical composition is used for treating cancers.

Another object of the present invention is to provide a pharmaceutical kit comprising:
at least one pharmaceutical composition comprising:
   at least one T cell clone expressing an endogenous CD16 receptor as described hereabove and/or
   modified T cells expressing an exogenous CD16-like receptor or at least one modified T cell clone expressing an exogenous CD16-like receptor, said CD16-like receptor being selected from the group consisting of CD16 receptors, CD16/γ chimeric receptors and CD16/ζ chimeric receptors, as described here above,
and at least one immuno-therapeutic agent such as:
   a tumor antigen selected from the group consisting of peptides derived from the MAGE, BAGE, GAGE and LAGE1/NY-ESO-1 gene families, and/or
   a monoclonal antibody selected from the group consisting in Infliximab, Basiliximab, Daclizumab, Trastuzumab, Rituximab, Ibritumomab tiutexan, Tositumomab, Gemtuzumab ozogamicin, Alemtuzumab.

Optionally associated with the kit can be included a notice in the form prescribed by a governmental agency regulating the manufacture, use or sale of pharmaceuticals or biological products, which notice reflects approval by the agency of manufacture, use or sale for human administration Instructions for use of the composition.

In a preferred embodiment, said kit further comprises at least one chemotherapeutic agent selected from the group consisting of etoposide, doxorubicin, vincristine, cyclophosphamide, prednisone, fludarabine.

### EXAMPLES

In the following description, all molecular biology experiments for which no detailed protocol is given are performed according to standard protocol.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Cell lines. For lentiviral production we used a 293 FT cell line (Invitrogen, Cergy Pontoise, France), a derivative of the 293F cell line that displays stable and constitutive expression of SV40 large T antigen under the control of the human CMV promoter. For retroviral production we used helper-virus-free Phoenix-Ampho packaging cells (G. P., Nolan, Standford, Calif.). 293 FT and Phoenix cell lines were maintained in high-glucose (4.5 g/liter) Dulbecco's modified Eagle's medium (DMEM) (Sigma Aldrich, St Quentin Fallavier, France) supplemented with 10% FBS (Biowest, Nuaillé, France) and 2 mM L-glutamine (Sigma Aldrich). Epstein Barr Virus B lymphoblastoid cell lines (BLCL) were derived from donor PBMC by in vitro infection using EBV-containing culture supernatant from the Marmoset B95.8 cell line from American Type Culture Collection (ATCC; Rockville, Md.) in the presence of 1 μg/ml cyclosporin-A. The Jurkat leukemia JRT3-T3.5 T cell line (the β-negative variant of Jurkat that lacks TCR expression, from ATCC) was grown in RPMI 1640 culture medium (Sigma Aldrich) supplemented with 10% FBS, 2 mM L-glutamine, penicillin (100 UI/ml), and streptomycin (0.1 μg/ml) (Biowest).

Construction of the FcγRIIIa/FcεRIγ chimeric gene encoding the CD16/γ receptor. cDNA coding for the extracellular domain of CD16 (nucleotides 1 to 651, GenBank Accession No.X52645) was amplified by PCR from a pcDNA3.1-FcγRIIIa (allotype V¹⁵⁸) plasmid kindly provided by Dr. M. Ohresser and Dr. H. Watier (EA 3853 Laboratoire d'Immunologie, Centre hospitalier Régional et Universitaire, Tours, France). cDNA for FcεRIγ (nucleotides 83 to 283, GenBank Accession No.BC033872) comprised a two amino-acid sequence (Pro-4-Gln5) of the extracellular domain and the intact transmembrane and intracytoplasmic domains as previously described [Vivier E, Rochet N, Ackerly M, et al. Int Immunol. 1992; 4:1313-1323]. FcεRIγ cDNA was amplified by RT-PCR using total RNA from cultured human NK cells and cloned in pcDNA3.1 (Invitrogen, Cergy Pontoise, France). Oligonucleotide primers (Sigma-Genosys, Saint Quentin Fallavier, France) used for the PCR reactions were as follows: CD16 sense: 5′ GCG GGATCC TCT TTG GTG ACT TGT CCA 3; CD16 anti-sense: 5′ GCG GAA TTC CCC AGG TGG AAA GAA TGA 3′; Gamma sense: 5′ CCCTG GAATTC CCT CAG CTC TGC TAT ATC 3′; Gamma anti-sense: 5′ CATCTA GCGGCCGCCTA CTG TGG TGG TTT C 3′. To generate the pcDNA3.1/FcγRIIIa/FcεRIγ, the 663-bp BamHI-EcoRI FcγRIIIa fragment was ligated into the pcDNA3.1/FcεRIγ plasmid. The sequence of FcγRIIIa/FcεRIγ chimeric construct was verified (Genome express, Meylan, France) and then cloned into a lentiviral LNT-sffv vector as well as into retroviral pMX vector.

Lentiviral vector production. LNT-sffv MCS was kindly provided by Dr Howe (Molecular Immunology Unit, Institute of Child Health, London, UK) [Demaison C, Parsley K, Brouns G, et al. Hum Gene Ther. 2002; 13:803-81340]. VSV-G pseudotyped vectors were produced by transient transfection of three plasmids into 293FT cells using the ViralPower Lentiviral Expression system (Invitrogen, Cergy Pontoise, France) [Dull T, Zufferey R, Kelly M, et al. J Virol. 1998; 72:8463-8471]. Three million 293FT cells were transfected by CaCl₂ precipitation with 12 μg plasmid: 9 μg viralPower Packaging Mix (pLP1, pLP2, pLP/VSVG) and 3 μg LNT-sffv/CD16-FcεRIγ. The medium (10 ml) was replaced 6 h after transfection and conditioned medium was collected 48 h post-transfection then filtered through 0.45-μm-pore-size filters. Viral particles were concentrated 100 fold by ultracentrifugation at 26,000 rpm for 90 min at 4° C. The viral pellet was resuspended in PBS and kept at −80° C. until use. Viral titer was determined by transduction of Jurkat T cells (1×10⁵ cells per well in 96-well plates) with serial dilutions of virus and analyzed for CD16 expression at 3 to 5 days post-infection. LNT-sffv/FcγRIIIa-FcεRIγ titers were typically 2-5×10⁷ (Infectious Units) IU/ml.

Retroviral vector production. CD16/γ cDNA was cloned into BamHI and NotI sites of the pMX vector [Onishi M, Kinoshita S, Morikawa Y, et al. Exp Hematol. 1996; 24:324-329]. Transient retroviral supernatants were produced by transfection of Phoenix-Ampho packaging cells. Two million Phoenix-Ampho cells were seeded in 10-cm-diameter dishes 24 h prior to transfection. Transfection was performed with 6 μg pMX/CD16/γ plasmid DNA using FuGENE 6 reagent (Roche, Meylan, France). Conditioned medium was collected 48 h post-transfection, filtered through 0.45-μm-pore-size filters and kept at −80° C. until use.

T cell clone transduction using lentiviral supernatant. Clone 18-DO259 is a CD4+ cytolytic EBV (peptide 23 EBNA2)-specific human T cell clone. Clone 4 and 31 are two CD4+ cytolytic HLA-DPB1*0401-specific human T cell clones. Clone 31-DO.8 is a CD8+ cytotoxic CMV (peptide N9V/pp65)-specific T cell clone. All clones were cultured using the following standard conditions: 1×10⁶ T cells were stimulated in 96-well U-bottomed plates in the presence of irradiated (35 Gy) pooled allogeneic feeder cells (1×10⁷ PBMC and 1×10⁶ BLCL), 1 μg/ml leukoagglutinin-A (Pharmacia, Upsalla, Sweden) and 300 UI/ml of recombinant IL-2 (Roussel-Uclaff, Romainville, France). Five days after stimulation, T cell clones were resuspended in RPMI1640 culture medium (Sigma Aldrich) supplemented with 8% human serum and 300 UI/ml of recombinant IL-2, seeded at 3×10⁵ cells in 450 μl per well in 24-well plates, and exposed to lentiviral supernatant corresponding to a multiplicity of infection (m.o.i) of 10 in the absence of polybrene. The culture medium was changed 24 h after infection. Mock (nontransduced) controls were performed in parallel, but in this case no viral supernatant was added to the T cell clones. Transduction efficiencies were assessed 5 days later.

Generation, expansion and transduction of EBV-specific cytotoxic cells using retroviral supernatant. Donor PBMC were plated in 24-well culture plates in RPMI1640 culture medium (Sigma-Aldrich) supplemented with 8% pooled human serum (HS), 1% L-glutamine, 100 U/ml penicillin and 0.1 μg/ml streptomycin at 2×10⁶ cells/well and stimulated with 5×10⁴ 35 Gray-irradiated autologous BLCL (PBMC:BLCL ratio of 40:1). After 10 days, T cells were collected and restimulated at a T:B ratio of 4:1 (5×10⁵ T cells and 1.25×10⁵ BLCL/well). IL-2 was added 3 days after the second stimulation. One day thereafter, retroviral transduction was performed by mixing 2×10⁶ T cells (in 2 ml RPMI, 8% HS supplemented with 80 UI IL-2/ml) with 2 ml of retroviral supernatant and 8 μg/ml polybrene and then centrifuging at 2400×g for 90 minutes at 34° C. As a control, the T cells were incubated with nontransfected Phoenix-ampho cell supernatant (nontransduced control CTL). The day after transduction half of the medium was changed. Transduction efficiencies were assessed 3 days later and a third stimulation was performed 7 days after the second, in the presence of IL-2 and with an identical T:B ratio (4:1).

Immuno-selection of transduced cells. Infected cells were analyzed by FACS after staining with a mouse-anti-human CD16 mAb (3G8) and immuno-selection using Goat anti-mouse-IgG1 coated beads (Dynabeads M-450, Dynal AS, Oslo, Norway) according to the supplier's instructions. Purity was >95% according to CD16 expression.

Cytotoxicity assay. Cytotoxic activity was assessed using a standard ⁵¹Cr release assay. Target cells were labeled with 100 μCi ⁵¹Cr for 1 h at 37° C., washed four times with culture medium, and then plated at the indicated effector-to-target cell ratio in a 96-well flat-bottom plate. An autologous BLCL was used as a model of autologous tumor and the humanized anti-CD20 mAb Rituximab (Roche, UK) was used (at 2 μg/ml) to induce ADCC. In some experiments, the anti-Her2/neu mAb Trastuzumab (Roche, UK) was used (at 10 μg/ml) as a control. For ADCC assays, the indicated monoclonal antibody was incubated with target cells for 20 min before addition of effector cells. In some experiments, autologous BLCL were loaded with the HLA-A2 binding peptide NLVPMVATV (referred to as N9V) derived from the pp65 CMV phosphoprotein. For loading, target cells were incubated for 30 min at 37° C. in the presence of different concentrations of peptides, and were washed twice in RPMI-FBS. After a 4 h incubation at 37° C., 25 μl of supernatant were removed from each well, mixed with 100 μl scintillation fluid, and ⁵¹Cr activity was counted in a scintillation counter. Each test was performed in triplicate. The results are expressed as the percentage of lysis, which is calculated according to the following equation: (experimental release-spontaneous release)/(maximal release-spontaneous release)×100, where experimental release represents the mean counts per minute (cpm) for the target cells in the presence of effector cells, spontaneous release represents the mean cpm for target cells incubated without effector cells, and maximal release represents the mean cpm for target cells incubated with 1% Triton X 100. For blocking experiments the F(ab′)2 fragment of the anti-human CD16 specific-mAb 3G8 (Coger, Paris, France) was added at a concentration of 10 μg/ml for the entire ADCC assay.

Phenotyping. The following mAbs and their isotype controls were used: anti-CD16 (3G8)-PE or -PC5, anti-CD3-FITC, anti-CD4-FITC and anti-CD8-FITC (Beckman Coulter, Roissy, France). Two hundred thousand (0.2×10⁶) cells were incubated for 10 min at RT in V-bottom microtiter plates in the presence of optimal concentrations of antibodies diluted with PBS supplemented with 5% human serum (HS) in a final volume of 25 μl. After staining, plates were centrifuged, the supernatant was discarded by flicking and wells were washed twice with 200 μl ice-cold PBS. Labeled cells were analyzed using a FACScan flow cytometer (Beckton Dickinson, Mountain View, Calif.).

In vitro stimulation of T cell clones. Stimulation of T cell clones was performed in 96-well flat-bottom plates at 10⁵ cells per well in 0.1 ml. In some experiments, 3300 BLCL per well were used as target cells (effector to target ratio=30:1) and humanized anti-CD20 (rituximab) (0.02 or 2 μg/ml) was used to induce ADCC. T cell clones were also incubated with different concentrations of soluble rituximab (1 to 1000 μg/ml). As a positive control, T cell clones were stimulated with 10 ng/ml phorbol myristate acetate (PMA) (Sigma) and 1 μg/ml ionomycin (sigma). Cells were cultured for 2 h at 37° C. in a humidified atmosphere of 5% CO2 in air. Brefeldin-A was then added at 10 μg/ml and the cells were cultured for an additional 4 h at 37° C. Cells were transferred into 96-well V-bottomed plates, pelleted, resuspended in PBS, washed once more, and resuspended in PBS-2% formaldehyde (Euromedex, Nundolsheim, France). Cells were then fixed for 15 min at room temperature. Fixed cells were washed twice in PBS and stored in PBS at 4° C. in the dark overnight.

Permeabilization and staining. Cells were pelleted and washed in 150 μl of 1X BD™ Phosflow Perm/Wash buffer (BD Biosciences Pharmingen, Le Pont de Claix, France) and resuspended in 50 μl of 1X BD™ Phosflow Perm/Wash buffer for 20 min at RT. The following monoclonal antibodies (mAbs) were used: PE-mouse anti-human TNFα (Mab 1, BD Biosciences Pharmingen), PE-mouse anti-human INFγ (B27, BD Biosciences Pharmingen) or with mouse IgG1 (BD Biosciences Pharmingen) as a negative control. Cells were stained at RT for 20 min with 50 μl of the aforementioned PE-mAbs diluted 1:50 in 1X BD™ Phosflow Perm/Wash buffer. The cells were then pelleted, washed in 1X BD™ Phosflow Perm/Wash buffer followed by two further washes in PBS. For flow-cytometric analysis data were collected and analyzed on a FACSscan flow cytometer (BD Biosciences Pharmingen).

Proliferation assay. More than 3 weeks after the last stimulation 2.5×10⁴ resting T cells were co-cultured (in triplicate) with 35 Gy-irradiated BLCL cells in 96-well flat-bottomed plates for 2 days at a responder to stimulator ratio of 1:1. Six hours before harvesting 1 μCi of ³H-thymidine was added to each well. ³H-thymidine uptake was then measured in a liquid p scintillation counter (Betaplate, Wallac Oy, Finland). Results are expressed as mean value for each triplicate.

Generation, expansion of EBV-specific cytotoxic T cells. Donor PBMCs were plated in 24-well culture plates in RPMI1640 (Sigma-Aldrich, Les Ulis, France) culture medium with 8% pooled human serum (HS) and futher supplemented with 1% L-glutamine, 100 U/ml penicillin and 0,1 μg/ml streptomycin at 2×10⁶ cells/well and stimulated with 5×10⁴ 35 Grays-irradiated autologous BLCL (PBMC:BLCL ratio of 40:1). After 10 days, T cells were collected and restimulated at T:B ratio of 4:1 (5×10⁵ T cells and 1,25×10⁵ BLCL/well). IL-2 was added 3 days after the second stimulation.

Expression vectors: Expression vectors encoding six lytic EBV proteins (BZLF1, BMLF1, BRLF1, BCRF1, BMRF1, and BHRF1), all of the latent EBV proteins (EBNA-1, -2, -3a, -3b, -3c, and -LP, LMP1, and LMP2) and the following HLA class I alleles: HLA-A*2401, HLA-B*4403, HLA-Cw*06, HLA-Cw*1601, were described previously [Scotet et al. J Exp Med 1996, November 1; 184(5):1791-800].

COS transfection and T cell stimulation assay: COS cell transfection was performed using the DEAE-dextran chloroquine method, as already described [Scotet et al. J Exp Med 1996, November 1; 184(5):1791-800; Scotet et al. Eur J Immunol 1999, March; 29(3):973-85; Brichard et al. J EXp Med 1993, August 1; 178(2):489-95]. Briefly, 15,000 COS cells were cotransfected with 100 ng of an expression vector coding for an EBV protein and 100 ng of an expression vector coding for one of the HLA class-I alleles. Transfected COS cells were used 48 h after transfection for CTL stimulation assays. T-cells to be tested (5×10⁴) were added to transfected COS cells. Culture supernatants were harvested 6 h later and tested for TNF-α content by measuring culture supernatant cytotoxicity for WEHI 164 clone 13 in a colorimetric assay [T. Espevik, J. Nissen-Meyer J Immunol Methods, 95(1986)99-105]. OD was calculated from duplicate samples. Values were considered significant when superior to two SD above the mean.

### Results

### I. T Cells Expressing an Endogenous CD16 Receptor

T Cells Coexpressing the Alpha-Beta T Cell Receptor (αβTCR) and the CD16 Receptor (FcγRIIIA) are Present in all Individuals in Number Comparable to that of T Lymphocytes Coexpressing the Gamma-Delta-T Cell Receptor (γδTCR) and CD16.

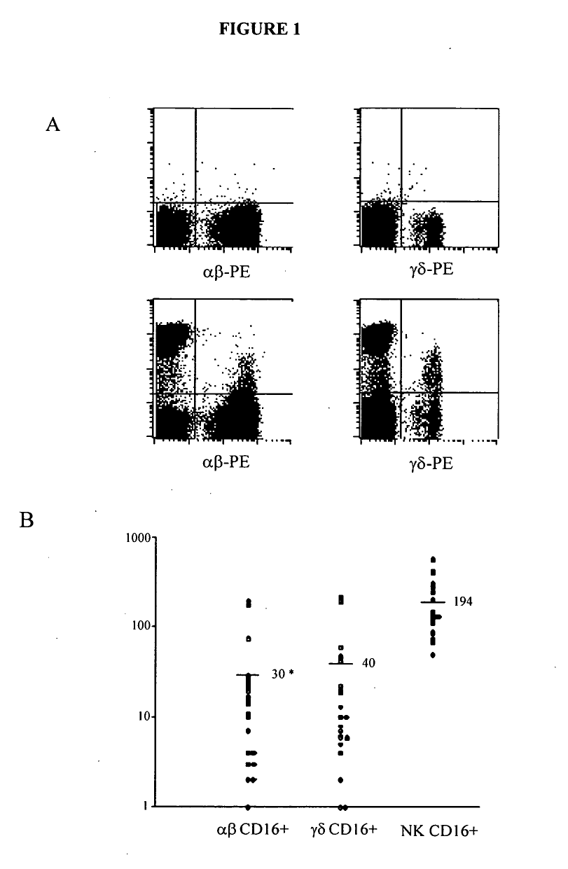

Peripheral blood mononuclear cells were stained with antibodies to αβTCR, γδTCR and CD16. Upon analysis of gated lymphocytes, three subsets of CD16 expressing cells were identified: CD16+ NK cells, CD16+ αβT-cells and CD16+ γδT-cells (FIG. 1A). Cytometric panels refer to a representative healthy donor. FIG. 1B shows the analysis of the absolute number of CD16+ NK cells, CD16+ αβT-cells and CD16+ γδT-cells in the peripheral blood of 30 healthy donors. * indicates the mean. This result indicates that a significant population of T cells expressing a CD16 receptor is present in all individuals.

CD16+ αβT-cells were further phenotypically characterized (Table I). Surface phenotype of CD16+ αβT-cells was identified by mAb in conjunction with three-color immunofluorescence tests: FITC-αβ TCR, PC5-CD16 and PE-different markers. Values indicate the percentage of positive cells. *Kirs=CD158a,h, CD158b and Kirp70.

TABLE 1

| | Donor no. | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| percent αβ+ CD16+ | 15 | 0.26 | 2.31 | 1.54 | 1.22 | 1.13 | 1.54 | 0.43 | 1.84 | 12.3 | 1.63 | 1.44 | 2.65 | 4.8 | 2.11 |
| among αβ+ | | | | | | | | | | | | | | | |
| CD4 | 57.5 | 22.0 | 14 | 5.2 | 1.7 | 17.9 | 1.7 | 14.7 | 8.5 | 12.0 | ND | 14.7 | 28.4 | 26.6 | 6.4 |
| CD8 | 65.8 | 89.0 | 99 | 96.8 | 97.6 | 98.0 | 99.3 | 92.7 | 97.0 | 91.7 | 81.0 | 85.2 | 81.0 | 93.9 | 71.6 |
| CD27 | 6.9 | ND | ND | 43.2 | 10.2 | 15.4 | 13.5 | 64.7 | 34.8 | 7.3 | ND | 11.9 | 62.4 | 8.2 | 20.8 |
| CD28 | 4.5 | 16.0 | 28 | 18.1 | 5.0 | 2.6 | 2.4 | 22.6 | 8.8 | 2.2 | ND | 9.3 | 44.9 | 5.6 | 10.1 |
| CD45RO | 60.4 | 18 | 29.8 | 47.2 | 5.0 | 41.7 | 3.3 | 30.8 | 88 | 72.6 | 59.0 | 10.1 | 61.2 | 92.7 | ND |
| CD45RA | 42.0 | ND | ND | 61.8 | 97.9 | 89.7 | 99.7 | 97.0 | 86.8 | 65.4 | 93.0 | 96 | 70.0 | 82.3 | 96.7 |
| CD57 | 84.8 | 65 | 85 | 64.0 | 89.2 | 91.3 | 66.4 | 36.6 | 89.8 | 92.3 | 50.0 | 84.2 | 46.9 | 92.1 | 70.6 |
| CD62L | 70.4 | 48 | 47.5 | 27.5 | 7.45 | 50.3 | 28.0 | 58.5 | 54.8 | 53.7 | ND | 48.0 | 53.8 | 48.2 | 34.3 |
| CCR7 | 1.1 | ND | ND | 21.1 | 0.12 | 0.5 | 0.2 | 12.5 | 5.3 | 1.2 | 47.0 | 2.7 | 20.2 | 8.6 | 6.3 |
| Kirs* | | ND | ND | 20.2 | 8.32 | 8.0 | 37.3 | 23.7 | 10.2 | 13.3 | ND | 43.0 | 16.5 | 23.1 | 44.9 |
| CD32 | ND | ND | ND | ND | ND | ND | ND | ND | ND | ND | ND | ND | 6.5 | 1.4 | 2.4 |
| CD64 | ND | ND | ND | ND | ND | ND | ND | ND | ND | ND | ND | ND | 1.2 | 0 | 1.5 |

T Cells Coexpressing the Alpha-Beta T Cell Receptor (αβTCR) and the CD16 receptor (FcγRIIIA) can be Cloned from Peripheral Blood Lymphocytes. The αβTCR CD16+ T-Cell Clone Retained CD16 Expression and Mediated ADCC During Long-Term Culture.

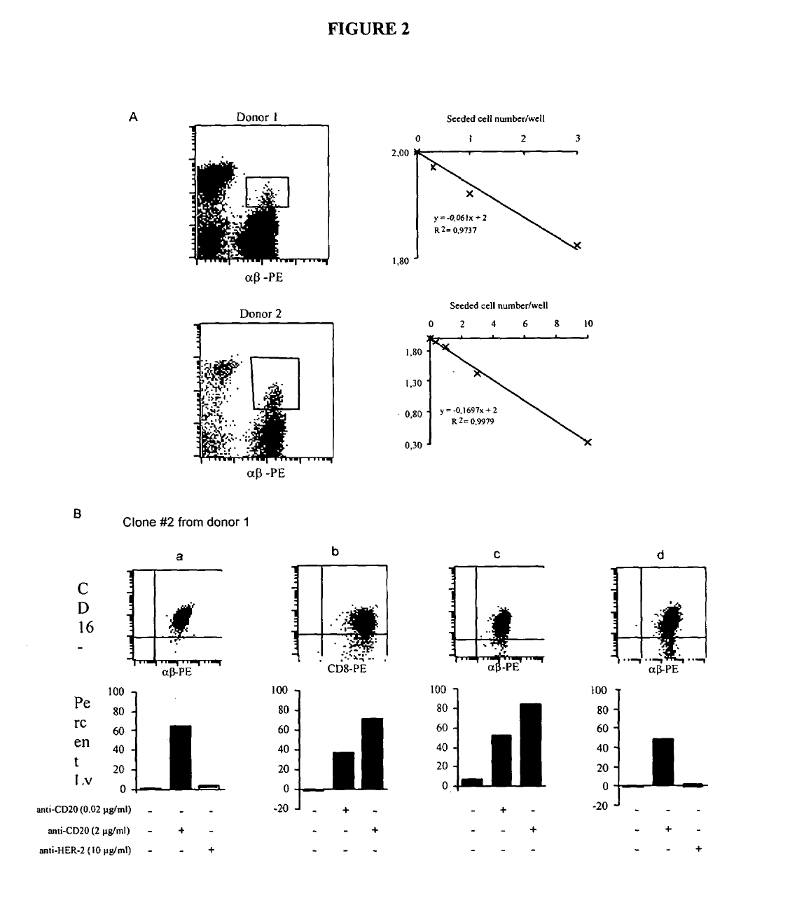

PBMC were stained with PE-anti-αβ antibody and PC5-anti-CD16 antibody. αβ CD16+ T-lymphocytes sorting was performed on a FACSVantage™ and cloned by limiting dilution using a non specific stimulation. Cloning efficiency were 0.75 and 0.30 (according to Poisson Distribution) (FIG. 2A).

FIG. 2B upper panel shows the maintenance of CD16 expression in CD 16+ αβ T-cell clone. T-cell clone was analysed by flow cytometry for CD16 expression over a 2.5 month period. a=Days 28 after cloning, b=Days 27 after the first non-specific stimulation, c=Days 52 after the first non-specific stimulation, d=After freezing and thawing, 38 days after the first stimulation.

In FIG. 2B lower panel, representative CD16+ αβ T-cell clone was tested for ADCC activity against 51Cr-labeled autologous BLCL, in presence of either rituximab (anti-CD20, 0.02 μg/ml or 2 μg/ml) or herceptin (anti-HER-2, 10 μg/ml) as a negative control. Results are expressed as percentage of specific lysis (effector-to-target ratio=30:1, mean of triplicate).

We therefore demonstrated that T cells expressing an endogenous CD16 receptor can be cloned and that these CD16+ T-cell clone are capable of retaining CD16 expression and mediating ADCC during long-term culture.

CD16+ αβ T-Cell Clone Produce Cytokines Only when the CD16 Molecule is Crosslinked in the Presence of mABs and Target Cells.

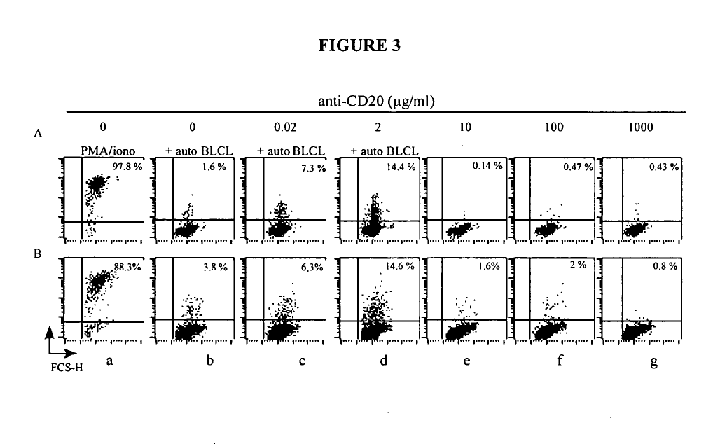

The CD16+/CD8+ T cell clone #14 from donor 1 (FIG. 3A) and the CD16+/CD4+ T-cell clone #21 from donor 2 (which doesn't recognizes the autologous BLC through its TCR) (FIG. 3B) produced TNFα after PMA+ionomycin stimulation (a) was activated only after CD16-crosslinking in the presence of the autologous BLCL and 0.02 or 2 μg/ml of anti-CD20 (b, c and d) but remained unstimulated by the soluble mAb at concentrations up to 1000 μg/ml (e,f,g).

EBV-Specific Cytotoxic T Cells Contain CD16+ αβ T Cells and Mediate ADCC.

EBV-specific CTLs were selected against the aulogous BLCL and stained with PE-anti-αβ antibody and PC5-anti-CD16 antibody. ADCC activity of the EBV-specific CTLs were evaluated against 51Cr-labeled allogeneic BLCL in presence of either rituximab (anti-CD20, 2 μg/ml) or herceptin (anti-HER-2, 10 μg/ml) as negative controls. Results are expressed as percentage of specific lysis (effector-to-target ratio=30:1, mean of triplicate). Therefore, these results show that EBV-specific cytotoxic T cells expressing an endogenous CD16 receptor are capable to mediate ADCC.

### II. Modified T Cells Expressing an Exogenous CD16-Like Receptor.

FcγRIIIα/FcεRIγ Vectors.

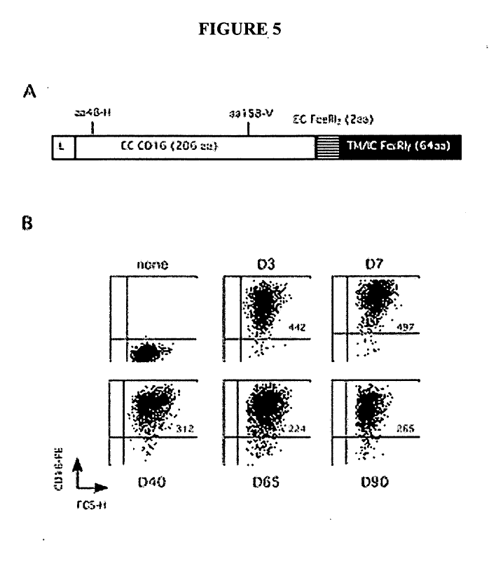

cDNA encoding the chimeric CD16/γ receptor, constructed as described in the Materials and Methods, comprised the peptide signal and the extracellular domain (except the last two amino acids) of CD16, two amino acids of the extracellular domain, as well as the full transmembrane and the full intracytoplasmic domains of the FcεRIγ (FIG. 5A). This construct was cloned into a lentiviral LNT-sffv vector [Demaison C, Parsley K, Brouns G, et al. Hum Gene Ther. 2002; 13:803-813], or into a retroviral pMX vector [Onishi M, Kinoshita S, Morikawa Y, et al. Exp Hematol. 1996; 24:324-329] and viral titers determined on the Jurkat cell line. Persistence of CD16/γ expression was evaluated on Jurkat cells after transduction using lentiviral supernatant, of which 98% were transduced after infection (FIG. 5B). CD16/γ expression was not detrimental to cell growth (data not shown) and after more than 3 months of culture, all cells still expressed high levels of CD16/γ molecules (FIG. 5B).

Generation of CD16/γ T Cell Clones.

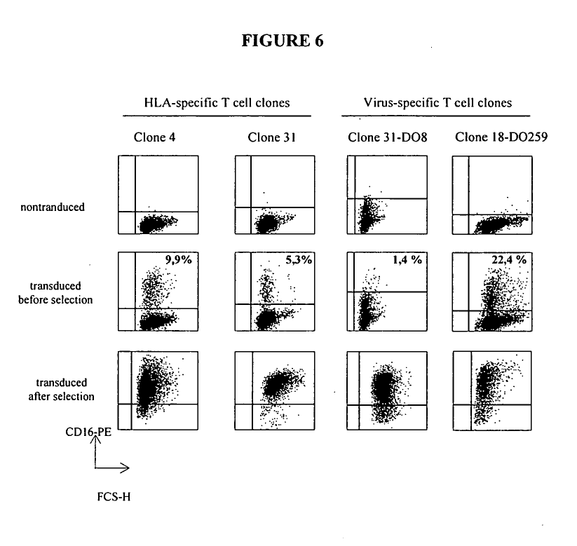

Four CD4+ and CD8+ antigen-specific T cell clones were exposed for 24 h to CD16/γ lentiviral vector supernatant. After 5 days, clones were monitored for CD16/γ expression by flow cytometry with an CD16-PE mAb. Transduction efficiencies ranged from 1.4% to 22.4% (FIG. 6). After immuno-selection using the 3G8 mAb, T cell clones were further analyzed and shown to retain CD16/γ expression at the same level during the entire follow-up period. In addition their CD3 expression remained identical to that observed in nontransduced T-cell clones (data not shown). Finally, the binding specificity of the human IgG isotypes for the T cell clones was similar in our hands to that observed for purified NK cells (IgG3>IgG1>IgG2>IgG4) and the binding was almost totally inhibited in the presence of saturating amounts of the anti-CD16 mAb 3G8 (data not shown).

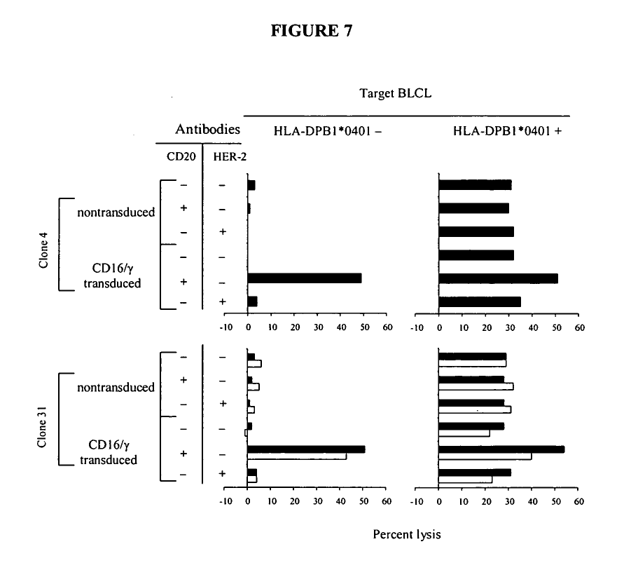

ADCC by Allospecific CD4+ T-Cell Clones Expressing CD16/γ Chimeric Molecules (FIG. 7).

Clone 4 and clone 31 are two allospecific HLA-DPB1*0401-specific T cell clones. The ADCC activity of transduced and nontransduced clones was evaluated using a standard 4 h ⁵¹Cr release assay. Target BLCL (all positive for CD20 and negative for Her2/neu antigens), that were either HLA-DPB1*0401 negative or positive, were coated or not with the humanized anti-CD20 mAb rituximab or the humanized anti-Her2/neu mAb trastuzumab as a negative control before coculture with the T cell clones.

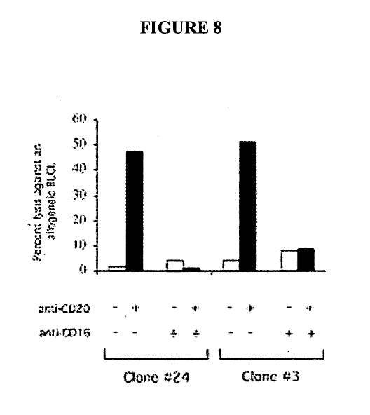

Cytotoxic activity of clones 4 and 31 against the HLA-DPB1*0401-positive BLCL, (the cognate target of their TCR), are shown on the right-hand panel of FIG. 7. In the absence of mAb able to recognize the BLCL (no mAb or anti Her2/neu), the cytotoxic scores of transduced or nontransduced T cell-clones were identical, strongly suggesting that TCR recognition was unaffected by CD16/γ transgene expression. In the presence of anti-CD20 mAb, only a slight increase in target cell lysis was observed, reflecting the fact that for these T cell clones, the cytotoxic activity was already almost maximal after TCR recognition. The cytotoxic scores against the HLA-DPB1*0401-negative BLCL are shown on the left-hand panel of FIG. 7. As expected, in the absence of mAb, the clones did not recognize the HLA-DPB1*0401-negative target cells. In contrast, both CD16/γ transduced clones killed the HLA-DPB1*0401 negative BLCL incubated with the anti-CD20 mAb. This cytotoxic activity was not observed in the presence of the anti-Her2/neu mAb. Finally, cytotoxic activity by CD16/γ-transduced T-cell clones was found to be inhibited in the presence of anti-CD16 mAb F(ab′)2-fragments (see FIG. 8). Thus, cytotoxicity was dependent on CD16 membrane expression on the T cell-clones and on target cell recognition by the mAb. Together, these data demonstrate that T cell-clones 4 and 31 had acquired the capacity to mediate ADCC after CD16/γ transduction. Interestingly, the cytotoxic activity of the transduced T cell clones against the HLA-DPB1*0401-positive BLCL and the HLA-DPB1*0401-negative BLCL in the presence of anti-CD20 mAb was similar. Thus, the co-engagement of TCR and CD16 was not cooperative in T cell clones. This observation is in line with a recent report showing that NKP46 engagement did not enhance CD16-dependant responses of NK cells [Bryceson Y T, March M E, Ljunggren H G, Long E O. Blood. 2006; 107:159-166] and supports the conclusion proposed by Bryceson et al. that ITAM-based signals do not enhance one another [Bryceson Y T, March M E, Ljunggren H G, Long E O. Blood. 2006; 107:159-166]. Altogether, the above results demonstrate that CD16/γ transduction enabled T-cell clones to recognize Ab-coated target cells in the absence of TCR recognition and that TCR recognition was not affected by CD16/γ transgene expression.

CD16-Crosslinking But not Soluble Mab Induced Thymidine Incorporation and Cytokine Production by CD16/γ-Transduced T Cell Clones.

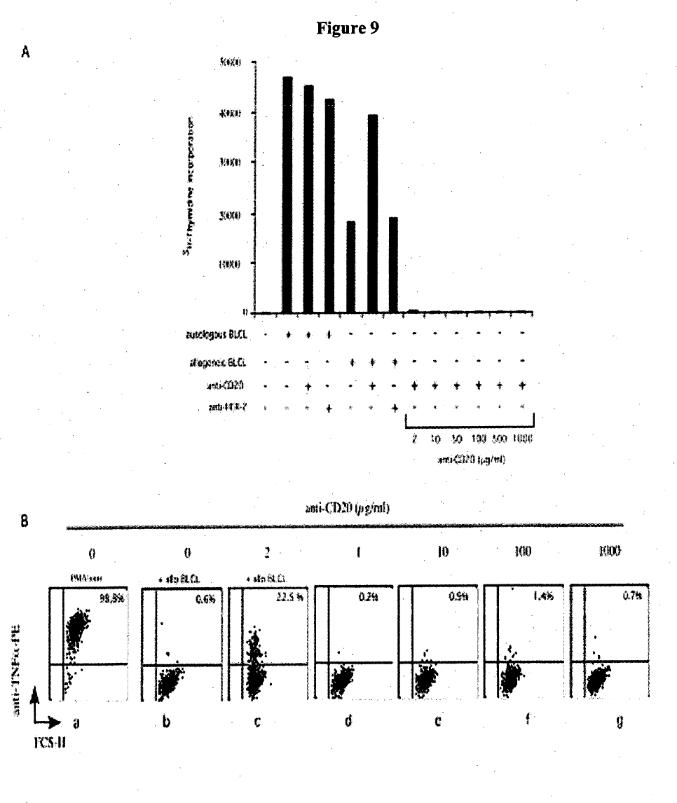

To test whether T cell responses other than cytotoxic activity could be initiated in CD16/γ transduced T cells, several T cell clones were tested for their ability to proliferate and produce cytokines (INFγ, TNFα and IL-2) after CD16 exposure to antibody coated cells. To exclude the possibility that soluble Ab can activate the clones, mAb concentrations of up to 1000 μg/ml were tested in the absence of target cells. Examples of results are presented in FIG. 9: the specific proliferation (against the autologous BLCL) of the CD8+ EBV-specific CD16/γ-transduced T cell clone #24 was unaffected by the presence of mAb against CD20 or HER-2. In contrast, against the allogeneic BLCL, the basic proliferation observed increased up to that observed against the specific target, in the presence of anti-CD20. This effect was not observed in the presence of anti-HER-2, suggesting that crosslinking was required to induce proliferation. Because the FcεRIγ signaling molecule was physically linked to the FcγRIIIa receptor, it was important to exclude the possibility that soluble Ab could stimulate the CD16/γ transduced T cells. To this end, in the absence of BLCL, soluble anti-CD20 was tested at concentrations of up to 1000 μg/ml. As shown in FIG. 9A, no thymidine incorporation was detected at any concentration tested. The same conclusions could be drawn for cytokine production: the results obtained for TNF production by clone #7 are presented in FIG. 9B. Essentially all cells from this clone were able to produce TNF when stimulated with PMA and Ca ionophore. Following crosslinking to target BLCL, 22,5% of cells from the clone became positive for TNF. In contrast, in the absence of target cells, the soluble mAb was unable to induce significant TNF production by the clone, at concentrations of up to 1000 μg/ml. Three independent experiments were performed with 3 different CD16/γ-transduced T cell clones and for three cytokines (TNFαc, IFNγ and IL-2), leading to the same conclusion. The same results were also observed when testing human serum at a concentration of up to 50%.

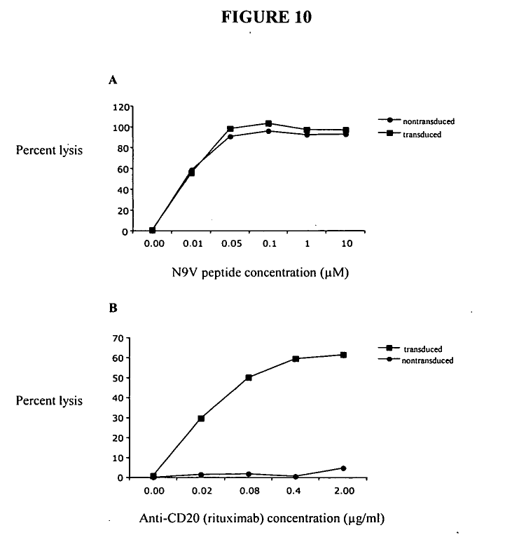

TCR and Antibody Dependant Recognition of the Target Cell by a CD16/γ Transduced CD8+ HLA-A*0201/CMV-pp65^{N9V} Specific T-Cell Clone (FIG. 10).

To assess more precisely whether CD16/γ transduction could affect TCR signaling, we transduced a CD8+ HLA-A*0201/CMV-pp65^{N9V}-specific C31DO8 T-cell clone. Non transduced and transduced C31DO8 clones were then tested against the autologous BLCL loaded with varying concentrations of the HLA-A2 binding peptide NLVPMVATV (referred to as N9V) derived from the pp65 CMV phosphoprotein. According to the results shown on the top panel of FIG. 10, BLCL lysis increased with increasing concentrations of N9V peptide and maximal lysis was achieved at the same peptide concentration (50 nM) for both clones, strengthening the previous observation with allospecific T cell-clones that CD16/γ transgene expression did not affect TCR signaling. To assess ADCC activity, the autologous target BLCL was incubated with varying concentrations of the humanized anti-CD20 mAb. Confirming our previous results, in the absence of TCR signaling (i.e. in the absence of the N9V peptide) the CD16/γ transduced C31DO8 cell clone was able to kill the BLCL in the presence of anti-CD20, according to a dose-response that reached a plateau at 2 μg/ml.

Transduced EBV-Specific CTLs Expressed the CD16/γ Transgene on Both CD4+ and CD8+ T Cell Subsets and Showed Increased Cytotoxic Activity Against the Autologous BLCL in the Presence of Anti-CD20.

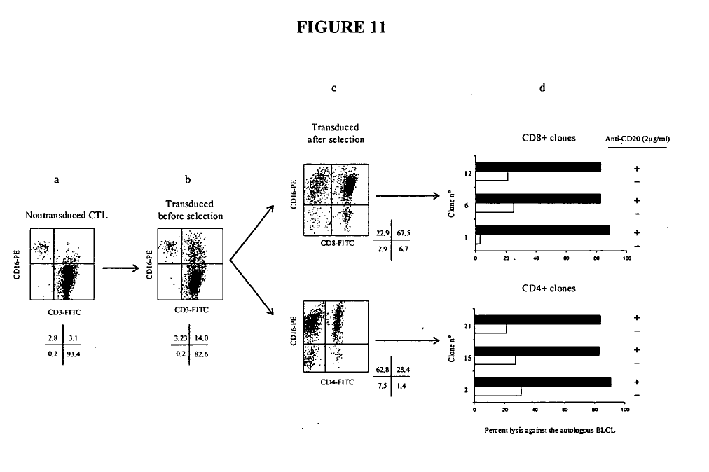

An EBV-specific CTLs were generated from a seropositive healthy donor and transduced with a retroviral pMX-CD16/γ supernatant (see Materials and Methods section). Flow cytometry analysis of CTLs stained with anti-CD16 specific antibody identified CD16 on 2.8% of the CTLs before transduction. These CD16+ lymphocytes were CD3—and thus corresponded to the few NK cells present in the CTL population (FIG. 11a). After transduction 14.0% of the CD3+ CTLs became CD16+ (FIG. 11b). Notably, the level of CD16 expression on the CD3+ CTLs was very similar to that observed on the NK cells (FIG. 11b). After immuno-selection, staining of the transduced CTLs with CD16-PE and CD4- or CD8-FITC mAb, revealed the presence of 28.4% CD4+ and 67.5% CD8+ cells among the CD16+ lymphocytes (FIG. 11c). These proportions were similar to those observed for nontransduced CTLs (20.4% and 77.9% respectively, data not shown), showing that transduction was just as efficient for CD8+ cells as for CD4+ cells. Because of the presence of NK cells in the polyclonal population after CD16 purification, a panel of T cell clones was derived from the CTLs and examples of their ability to kill the autologous BLCL in the presence or absence of anti-CD20 are shown in FIG. 11d. For these CD8+ and CD4+ clones, which had a relatively low cytotoxic activity against the autologous BLCL, a large increase in their ability to kill the target BLCL was observed when the BLCL was coated with anti-CD20. For these clones when both the TCR and CD16/γ chain molecule recognize the same target the increased in cytotoxicity appeared different to that observed for the allospecific T cell clones presented in FIG. 7. The third CD8 clone in FIG. 11c was presented as an example of a non specific T cell (often present in various proportions in such polyclonal cultures) that became an effector against the BLCL in the presence of anti-CD20. Hence, transduction of the CD16/γ chimeric receptor in polyclonal EBV-specific CTLs confers ADCC potential to both the CD4+ and CD8+ T cell subsets.

1. A method for enhancing ADCC comprising the administration of an effective amount of T cells expressing a CD16-like receptor in an individual in need thereof, wherein said CD16-like receptor is selected from the group consisting of CD16 receptors, CD16/γ chimeric receptors and CD16/ζ chimeric receptors.

2. The method according to claim 1, wherein said T cells expressing a CD16-like receptor are natural T cells expressing endogenous CD16 receptor.

3. The method according to claim 1, wherein said T cells expressing a CD16-like receptor are modified T cells expressing an exogenous CD16-like receptor.

4. The method according to claim 1, wherein said effective amount of T cells expressing a CD16-like receptor is administrated via a parenteral route.

5. The method according to claim 4, wherein said effective amount of T cells expressing a CD16-like receptor is administrated intravenously.

6. The method according to claim 1, further comprising the administration of at least one immuno-therapeutic agent such as tumor antigens or monoclonal therapeutic antibodies.

7. The method according to claim 6, wherein said immuno-therapeutic agent comprises at least one tumor antigen selected from the group consisting of peptides derived from the MAGE, BAGE, GAGE and LAGE1/NY-ESO-1 gene families.

8. The method according to claim 6, wherein said immuno-therapeutic agent comprises at least one monoclonal therapeutic antibody selected from the group consisting of Infliximab, Basiliximab, Daclizumab, Trastuzumab, Rituximab, Ibritumomab tiutexan, Tositumomab, Gemtuzumab ozogamicin, Alemtuzumab.

9. The method according to claim 1, further comprising the administration of at least one immuno-depleting agent comprising at least one chemotherapeutic agent selected from the group consisting of 5-fluorouracil, aziathioprine, cyclophosphamide, anti-metabolites (such as fludarabine), anti-neoplastics (such as etoposide, doxorubicin, methotrexate, vincristine), prednisone, carboplatin, cis-platinum and the taxanes such as taxol.

10. The method according to claim 1 for treating cancer, optionally in combination with antitumoral vaccination.

11. The method according to claim 1 for treating cancer, especially solid tumors, optionally in combination with monoclonal antibody therapy.

12. The method according to claim 1 for treating cancer, especially haematological tumors, optionally in combination with monoclonal antibody therapy.

13. The method according to claim 1 for treating and/or preventing autoimmune diseases, optionally in combination with monoclonal antibody therapy.

14. The method according to claim 1 for treating tissue graft or organ rejection, including graft versus host disease, optionally in combination with monoclonal antibody therapy.

15. The method according to claim 1 for treating and/or preventing infectious diseases.

16. A T cell clone expressing an endogenous CD16 receptor.

17. The T cell clone according to claim 16, further expressing a TCR of known specificity.

18. The T cell clone according to claim 17, expressing a TCR directed against a virus selected from the group consisting of Epstein Barr viruses (EBV), cytomegaloviruses (CMV), human papilloma viruses (HPV) and herpes simplex virus (HSV1, HSV2).

19. The T cell clone according to claim 17, expressing a TCR directed against HLA molecules.

20. The T cell clone according to claim 17, further expressing a specific HLA combination, that is widespread in the recipient individuals.

21. A method for isolating a T cell clone expressing endogenous CD16 receptor, comprising:
isolating T cells expressing an endogenous CD16 receptor from PBL,
purifying said T cells expressing an endogenous CD16 receptor,
cloning T cells expressing an endogenous CD16 receptor,
optionally further expanding the at least one T cell clone thus obtained.

22. A method for producing a T cell clone expressing endogenous CD16 receptor, a TCR of known specificity, and optionally expressing a specific HLA combination that is widespread in the recipient individuals, comprising:
isolating and expanding at least one (known-antigen)-specific T cell optionally expressing a specific HLA combination that is widespread in the recipient individuals,
cloning said (known-antigen)-specific T cell,
isolating at least one (known-antigen)-specific T cell clone expressing an endogenous CD16 receptor,
optionally purifying said (known-antigen)-specific T cell clone expressing an endogenous CD16 receptor,
and optionally expanding said (known-antigen)-specific T cell clone expressing an endogenous CD16 receptor.

23. A pharmaceutical composition comprising at least one T cell clone expressing an endogenous CD16 receptor.

24. The pharmaceutical composition according to claim 23, further comprising a pharmaceutically acceptable carrier.

25. The pharmaceutical composition according to claims 23 for treating diseases or conditions requiring an ADCC enhancement selected from the group consisting of cancers, autoimmune diseases, tissue graft or organ rejections, bacterial or viral infections.

26. The pharmaceutical composition according to claims 23, wherein said T cell clone expresses a TCR of known affinity.

27. The pharmaceutical composition according to claims 26, wherein said TCR of known affinity is directed against a virus selected from the group consisting of Epstein Barr viruses (EBV), cytomegaloviruses (CMV), human papilloma viruses (HPV), and herpes simplex virus (HSV1, HSV2).

28. The pharmaceutical composition according to claims 26, wherein said TCR of known affinity is directed against HLA molecules.

29. The pharmaceutical composition according to claims 26, wherein said T cell clone further expresses a specific HLA combination, that is widespread in the recipient individuals.

30. A modified T cell expressing an exogenous CD16-like receptor, said CD16-like receptor being selected from the group consisting of CD16 receptors, CD16/γ chimeric receptors and CD16/ζ chimeric receptors.

31. The modified T cell according to claim 30, wherein the exogenous CD16-like receptor of the modified T cell is a CD16/γ chimeric receptor.

32. The modified T cell according to claim 30, being a T cell clone.

33. The modified T cell clone according to claim 32, wherein the exogenous CD16-like receptor of the modified T cell is a CD16/γ chimeric receptor.

34. The modified T cell according to any one of the claims 30 or 32, expressing a TCR of known specificity.

35. The modified T cell according to claim 34, wherein said TCR specificity is directed against a virus selected from the group consisting of Epstein Barr viruses (EBV), cytomegaloviruses (CMV), human papilloma viruses (HPV) and herpes simplex virus (HSV1, HSV2).

36. The modified T cell according to claim 34, wherein said TCR specificity is directed against HLA molecules.

37. The modified T cell according to claim 34, further expressing a specific HLA combination that is widespread in the recipient individuals.

38. A method for producing modified T cells expressing an exogenous CD16-like receptor, comprising:
isolating T cells,
transfecting or transducing said T cells to allow expression at their surface of an exogenous CD16-like receptor,
purifying modified T cells expressing an exogenous CD16-like receptor thus obtained,
optionally cloning at least one of the modified T cells expressing an exogenous CD16-like receptor,
and optionally further expanding the T cell clones thus obtained.

39. A method for producing modified T cells expressing exogenous CD16-like receptor, a TCR of known specificity, and optionally expressing a specific HLA combination that is widespread in the recipient individuals, comprising:
isolating and expanding at least one (known-antigen)-specific T cell optionally expressing a specific HLA combination that is widespread in the recipient individuals,
transfecting or transducing said (known-antigen)-specific T cell to allow expression at their surface of an exogenous CD16-like receptor,
isolating said (known-antigen)-specific T cell expressing an exogenous CD16-like receptor,
optionally cloning (known-antigen)-specific T cell expressing an exogenous CD16-like receptor,
optionally purifying said (known-antigen)-specific T cell clone expressing an exogenous CD16-like receptor,
and optionally expanding said (known-antigen)-specific T cell clone expressing an exogenous CD16-like receptor.

40. A viral vector comprising a gene encoding a CD16-like receptor, said CD16-like receptor being selected from the group consisting of CD16 receptors, CD16/γ chimeric receptors and CD16/ζ chimeric receptors.

41. The viral vector according to claim 40 being a lentivirus.

42. A composition comprising at least one modified T cell expressing an exogenous CD16-like receptor, said CD16-like receptor being selected from the group consisting of CD16 receptors, CD16/γ chimeric receptors and CD16/ζ chimeric receptors.

43. The composition according to claim 42, wherein said modified T cell is a T cell clone.

44. The composition according to any one of the claims 42 or 43, wherein said modified T cell expresses a TCR of known affinity.

45. The composition according to claim 44, wherein said TCR is directed against a virus selected from the group consisting of Epstein Barr viruses (EBV), cytomegaloviruses (CMV), human papilloma viruses (HPV) and herpes simplex virus (HSV1, HSV2).

46. The composition according to claim 44, wherein said TCR is directed against HLA molecules.

47. The compositiori according to claim 44, wherein said modified T cell expresses a specific HLA combination, that is widespread in the recipient individuals.

48. A pharmaceutical composition comprising at least one modified T cell expressing an exogenous CD16-like receptor, said CD16-like receptor being selected from the group consisting of CD16 receptors, CD16/γ chimeric receptors and CD16/ζ chimeric receptors.

49. The pharmaceutical composition according to claim 48, further comprising a pharmaceutically acceptable carrier.

50. The pharmaceutical composition according to claim 48, for treating diseases or conditions requiring an ADCC enhancement selected from the group consisting of cancers, autoimmune diseases, tissue graft or organ rejections including GVHD, bacterial or viral infections.

51. The pharmaceutical composition according to claim 48, wherein said modified T cell is a T cell clone.

52. The pharmaceutical composition according to any one of the claims 48 or 51, wherein said modified T cell expresses a TCR of known specificity.

53. The pharmaceutical composition according to claim 52, wherein said TCR specificity is directed against a virus selected from the group consisting of Epstein Barr viruses (EBV), cytomegaloviruses (CMV), human papilloma viruses (HPV) and herpes simplex virus (HSV1, HSV2).

54. The pharmaceutical composition according to claim 52, wherein said TCR specificity is directed against HLA molecules.

55. The pharmaceutical composition according to claim 52, wherein modified T cell further expresses a specific HLA combination, that is widespread in the recipient individuals.

56. A kit comprising:
at least one pharmaceutical composition comprising:
at least one T cell clone expressing an endogenous CD16 receptor, and/or
modified T cells expressing an exogenous CD16-like receptor or at least one modified T cell clone expressing an exogenous CD16-like receptor, said CD16-like receptor being selected from the group consisting of CD16 receptors, CD16/γ chimeric receptors and CD16/ζ chimeric receptors,
and at least one therapeutic agent such as:
a tumor antigen selected from the group consisting of peptides derived from the MAGE, BAGE, GAGE and LAGE1/NY-ESO-1 gene families, and/or
a monoclonal antibody selected from the group consisting of Infliximab, Basiliximab, Daclizumab, Trastuzumab, Rituximab, Ibritumomab tiutexan, Tositumomab, Gemtuzumab ozogamicin, Alemtuzumab.

57. The kit according to claim 56, wherein said T cell clone expressing an endogenous CD16 receptor, expresses a TCR of known affinity and optionally a specific HLA combination, that is widespread in the recipient individuals.

58. The kit according to claim 56, wherein said modified T cell clone expressing an exogenous CD16-like receptor, expresses a TCR of known affinity and optionally a specific HLA combination, that is widespread in the recipient individuals.

59. The kit according to claim 56, further comprising at least one chemotherapeutic agent selected from the group consisting of etoposide, doxorubicin, vincristine, cyclophosphamide, prednisone, fludarabine.


SEQUENCE LISTING (4 sequences)

SEQ ID NO: 1 - DNA, 27 bp, Artificial sequence
  CD16 sense primer
gcgggatcctctttggtgacttgtcca

SEQ ID NO: 2 - DNA, 27 bp, Artificial sequence
  CD anti sense primer
gcggaattccccaggtggaaagaatga

SEQ ID NO: 3 - DNA, 29 bp, Artificial sequence
  Gamma sense primer
ccctggaattccctcagctctgctatatc

SEQ ID NO: 4 - DNA, 30 bp, Artificial sequence
  Gamma anti sense primer
catctagcggccgcctactgtggtggtttc